(19) United States
(12) Patent Application Publication
(10) Pub. No.: US 2019/0091346 A1
(43) Pub. Date: Mar. 28, 2019

(54) PHOTOLABILE COMPOSITIONS AS A STABILIZATION PLATFORM

(71) Applicant: Nanoly Bioscience, Inc., Denver, CO (US)

(72) Inventors: Balaji V. Sridhar, Denver, CO (US); Mark W. Tibbitt, Denver, CO (US); Oyvind Hatlevik, Denver, CO (US); Jacob W. Heaps, Denver, CO (US); John Janczy, Denver, CO (US)

(73) Assignee: Nanoly Bioscience, Inc., Denver, CO (US)

(21) Appl. No.: 16/205,024

(22) Filed: Nov. 29, 2018

Related U.S. Application Data
(63) Continuation-in-part of application No. PCT/US2017/033860, filed on May 22, 2017.
(60) Provisional application No. 62/343,730, filed on May 31, 2016.

(51) Int. Cl. A61K 47/69; A61K 41/00; A61K 47/64

(52) U.S. Cl. CPC A61K 47/6903; A61K 47/645; A61K 41/0042

(57) ABSTRACT

A photodegradable polymeric system is presented that can be employed to entrap and stabilize bioactive therapeutics such as proteins and vaccines from environmental stressors. This system would obviate the need for refrigeration and would decrease transportation and storage costs of temperature-sensitive therapeutics. By using a photosensitive release system, users can administer temperature-sensitive compounds at their discretion. This photodegradable system will also permit the potential to stabilize temperature-sensitive therapeutics in a liquid suspension, eliminating the need for reconstitution.

### CROSS-REFERENCES TO RELATED APPLICATIONS

This application is a continuation-in-part of PCT application no. PCT/US2017/033860, filed May 22, 2017, which claims the benefit of priority to U.S. Provisional Application 62/343,730, filed on May 31, 2016, the contents of which are hereby incorporated by reference in the entirety for all purposes.

### BACKGROUND OF THE INVENTION

There is a growing need for many temperature-sensitive therapeutics such as vaccines to be stabilized from environmental stressors such as high temperature. Refrigeration and lyophilization are too cost prohibitve and have limited access to many people worldwide for life-saving medicines. Utilizing a synthetic material to circumvent the need for refrigeration would greatly reduce costs and increase access of therapeutics to millions worldwide. A critical aspect of designing a biomaterial to thermally stabilize relevant therapeutics is tuning and controlling the degradation behavior of materials.

Conventional degradation technology uses hydrolysis and/or enzymatic degradation, which are sustained processes that offer minimal spatial or temporal control. Most synthetic biomaterials degrade via hydrolysis, which can occur throughout the bulk or only at the surface of a biomaterial and leads to a sustained and non-instantaneous mass loss, which may be undesirable. Current photopolymerization and photodegradation techniques require the use of a photosensitizer, and often have no spatial control.

There is a need for an improved degradation process that allows for increased user control and the ability to thermally stabilize therapeutics in a liquid solution. The present invention satisfies these and other needs.

### BRIEF SUMMARY OF THE INVENTION

In one embodiment, the present invention provides compositions and methods that enable stabilization of temperature-sensitive therapeutics and biologics with a photodegradable polymeric system (“hydrogel”) using mono- and multifunctional macromolecular monomers (“macromers”) that degrade via single- and multi-photon photolysis mechanisms over a broad range of wavelengths. Due to the capability to tune the pore size of the hydrogel network, encapsulated therapeutics can be released in a depot fashion by passive diffusion in vivo. The macromers can form or be incorporated into hydrogel networks via covalent, non-covalent and/or ionic interactions. These hydrogel networks can controllably degrade both spatially and temporally.

More specifically, the present invention provides a photodegradable composition, comprising: (a) a photolabile group; (b) a polymeric backbone structure comprising one or more repeating units that may be the same or different; (c) a linker comprising repeating units of amino acid monomers joined by peptide bonds or a linker comprised of PEG, through which the backbone structure is attached to the photolabile group; (d) one or more reactive end groups at one or more ends of the linker, backbone, and/or photolabile group; optionally, (e) one or more therapeutic agents; and, optionally (f) one or more caged groups.

In another embodiment, the present invention provides polymers and networks incorporating macromers or hydrogels of the invention and optionally other substituents such as other polymeric structures.

In addition, the present invention provides a method of controlled degradation of a polymer comprising: providing a photodegradable polymer as described herein and exposing the photodegradable polymer to photoradiation of the appropriate wavelength and intensity to cause one or more of the photodegradable groups to photodegrade.

These and other aspects, objects and advantages will become more apparent when read with the figures and detailed description which follows.

### BRIEF DESCRIPTION OF THE DRAWINGS

FIG. 1 shows some different examples of structures of the invention.

FIG. 2 shows a general depiction of the formation and cleavage of networks of macromers of the invention.

FIG. 3 shows an embodiment and description of the release of a therapeutic agent.

FIG. 4 shows photodegradable network fabrication, wherein a tetra-functional PEG-DBCO is reacted with a diazide peptide crosslinker, which contains a nitrobenzyl ether photolabile moiety.

FIG. 5 shows how the photodegradable material can be used in both a gel suspension or liquid suspension to thermally stabilize vaccines and be photodegraded prior to administration.

FIG. 6 shows alkaline phosphatase (ALP) stored at 60° C. for four weeks, with and without the protection of the invention. Compared to a control sample stored at −70° C., retention of enzymatic activity was ˜5% for the sample stored without encapsulation and ˜80% for the sample stored with encapsulation and subsequently photoreleased.

FIG. 7 shows ligation activity of T4 DNA ligase stored at 40° C. for 24 hours on a λ DNA HindIII digestion. The ligation products are separated and visualized on a 1% TAE/agarose gel using ethidium bromide staining. Lane 1 contains a 1 kb DNA ladder; lane 2 contains λ/HindIII digestion fragments (no ligase added); lane 3 contains λ/HindIII+50U ligase as received from the manufacturer; lane 4 contains λ/HindIII+NanoShield encapsulated ligase stored at 40° C.; lane 5 contains λ/HindIII+NanoShield encapsulated ligase stored at −20° C.; lane 6 contains λ/HindIII+unencapsulated ligase (control ligase) stored at 40° C.

FIG. 8 shows ligation activity of T4 DNA ligase stored at 60° C. for 30 minutes on a λ DNA HindIII digestion. The ligation products are separated and visualized on a 1% TAE/agarose gel using ethidium bromide staining. Lane 1 contains a 1 kb DNA ladder; lane 2 contains λ/HindIII digestion fragments (no ligase added); lane 3 contains λ/HindIII+50U ligase as received from the manufacturer; lane 4 contains λ/HindIII+NanoShield encapsulated ligase stored at 60° C.; lane 5 contains λ/HindIII+NanoShield encapsulated ligase stored at −20° C.; lane 6 contains λ/HindIII+unencapsulated ligase (control ligase) stored at 60° C.; lane 7 contains λ/HindIII digestion fragments (no ligase added).

FIG. 9 shows enzymatic activity for beta-galactosidase (βGal) stored at 60° C. for two weeks and four weeks, with and without the protection of the invention. Compared to a control sample stored at 4° C., retention of enzymatic activity was ˜3% for the samples stored without encapsulation and >80% for the samples stored with encapsulation and subsequently photoreleased.

FIG. 10 shows percent maximum response for anti-CD3 antibody (1 mg/mL) stored at 65° C. for 1 hour, with the protection of the invention (NanoShield, dried sample) and without the protection of the invention (PBS, non-dried sample).

FIG. 11 shows percent maximum response for anti-CD3 antibody (1 mg/mL) stored at 65° C. for 72 hours, with the protection of the invention (NanoShield, dried sample) and without the protection of the invention (PBS, non-dried sample).

FIG. 12 shows percent maximum response for anti-CD3 antibody (0.1 mg/mL) after being subjected to five freeze/thaw cycles, with the protection of the invention (NanoShield) and without the protection of the invention (PBS+Trehalose). Both protected and unprotected anti-CD3 samples were dried prior to freeze/thaw cycles.

FIG. 13 shows percent maximum response for anti-CD3 antibody (1 mg/mL) after being subjected to five freeze/thaw cycles, with the protection of the invention (NanoShield) and without the protection of the invention (PBS+Trehalose). Dried and non-dried samples of protected anti-CD3 and unprotected anti-CD3 were subjected to freeze/thaw cycles.

FIG. 14 shows in vivo activity of ovalbumin (OVA) stored at 60° C. for 7 days and 14 days with and without the protection of NanoShield. Immunizations were prepared with stressed OVA (with or without NanoShield), or freshly prepared Ova with alum (40 μg/injection) at a final volume of 200 μL in sterile PBS. Groups of 5 mice were injected i.p. with the ova immunizations. Serum was collected weekly for 6 weeks via r.o. bleeds. IgG1 titers were determined by ELISA (minimum dilution 1:400).

### DETAILED DESCRIPTION OF THE INVENTION

### I. Definitions

“Photodegradable group” or “photolabile group” or “photorelease group” as used herein all refer to groups that break one or more bonds in response to exposure to radiation of the appropriate wavelength and energy. Such groups include the following: “photolabile nitrobenzo group,” “coumarin-azide cinnamic photorelease group,” and “coumarin cinnamic photorelease group.” The appropriate wavelength and energy is easily determinable by one of ordinary skill in the art without undue experimentation such as by the use of an absorbance spectrum to determine what wavelength(s) will cause photodegradation. The degradation of the photolabile group does not need a photosensitizer, although a photosensitizer may be used if desired. The use of the invention with a photosensitizer is easily performed by one of ordinary skill in the art without undue experimentation. Single- or multi-photon photolysis can be used to photodegrade the photolabile group. A broad range of wavelengths may be used for photodegradation, for example, those wavelengths in the ultraviolet spectrum, visible and infrared spectrum (between about 180 nm and 1.5 μm, for example) and all individual values and ranges therein, including UV-A (between about 320 and about 400 nm); UV-B (between about 280 and about 320 nm); and UV-C (between about 200 and about 280 nm). Other useful ranges include the radiation from visible, near-IR and IR lasers (about 500 nm to about 1.5 μm). All individual wavelengths and all intermediate ranges therein are intended to be included in this disclosure as if they were each listed separately. In some cases, at least one reactive end group is attached to photolabile group, either directly or indirectly through a linker or a polymeric backbone. The hydrogels can also degrade in vivo and release encapsulated therapeutics via passive diffusion.

“Reactive end groups” as used herein means those groups that are polymerizable by cationic, anionic, coordination, free-radical, condensation, bioorthogonal (i.e., click reactions), and/or other reactions as known in the art such as a pseudo-Michael addition. At least one reactive end group is incorporated into the photodegradable composition through attachment to one or more of the following components: the photolabile group, the polymeric backbone, or the linker. The reactive end groups may also form polymers through ionic interactions, self-assembly or non-covalent interactions, as known in the art. There are many reactive end groups known in the art. All reactive end groups that function in the macromers and polymers of the invention are intended to be included in this disclosure, even if not specifically mentioned. Some examples of reactive end groups include: acrylate, methacrylate, styrene, allyl ether, vinyl ether, isocyanate, cyanoacrylate, norbornylene, azide, dibenzocyclooctene (DBCO), triazide, phosphazine, imine, oxazoline, propylene sulfide, thiol groups, groups polymerizable using condensation reactions as known in the art, alkene, alkyne, “click” chemistry, carboxylic acid, epoxide, isocyanate, and other polymerizable groups known in the art (such as those produced by condensation of carboxylic acids with alcohols or amines to form polyesters or polyamides). Polymerization using reactive end groups is well-known in the art. Click chemistry (developed in the Sharpless group at The Scripps Research Institute) can utilize the copper (I) triazole formation from alkynes and azides, a highly efficient reaction (Wang, Q.; Chan, T. R.; Hilgraf, R.; Fokin, V. V.; Sharpless, K. B.; Finn, M. G. “Bioconjugation by Copper(I)-Catalyzed Azide-Alkyne [3+2] Cycloaddition”, J. Am. Chem. Soc. 2003, 125, 3192). There is growing interest in “click” chemistry in many applications; the triazole products can associate with biological agents through dipole interactions and hydrogen bonding (Kolb, H. C.; Sharpless, K. B. “The Growing Impact of Click Chemistry in Drug Discovery” Drug Discov. Today 2003, 8(24), 1128-1137). Other types of click reactions useful for the invention, which do not require the use of a copper catalyst, include the following reactions well known in the art: strain-promoted azide-alkyne cycloaddition (SPAAC), strain-promoted alkyne-nitrone cycloaddition (SPANC), alkene and azide [3+2] cycloaddition, alkene and tetrazine inverse-demand Diels-Alder, and alkene and tetrazole photoclick reactions.

The “polymeric backbone structure” or “backbone structure” as used herein mean a structure comprising any repeating unit into which a photodegradable group or photolabile group can be attached. There are many repeating units known in the art. All repeating units that function in the macromers and polymers of the invention are intended to be included in this disclosure, even if not specifically mentioned. Some examples of useful repeating units include poly(ethylene glycol) (PEG), poly(ethylene oxide), poly(vinyl alcohol), poly(vinylpyrrolidone), poly(ethyloxozoline), poly(ethylene oxide)/poly(propyleneoxide) block copolymers, polysaccharides, poly(hydroxylethylmethacrylates), poly(urethanes), poly(hydroxyethylacrylates), collagen, poly α-hydroxyesters, carbohydrates, proteins, poly(oxazoline), polyamino acids, poly(lactides), poly(styrenes), poly(acrylates), poly(methacrylates), poly(vinylethers), polyethylenes, poly(ethylene imine)s, polyesters, polypropylenes, —O—CH₂—CH₂C(O)NH—(CH₂CH₂O)ₙ—NH—C(O)CH₂—CH₂—O— (n=1-100), or any other polymer known in the art, and combinations thereof. Some backbones that are particularly useful for the invention include poly(ethylene glycol) (PEG), poly(styrene), poly(acrylate), poly(methacrylate), and poly(vinyl ether). The backbone can contain two or more different repeating units in any sequence, including random, gradient, alternating or block. The repeating units may be amphiphilic with respect to each other, the photodegradable group, the reactive end group and any other group in the macromer. The backbone structure can be a linear or branched chain of repeating groups. In some cases, the linear or branched backbone structure can be a “multi-arm backbone structure,” including 3-arm backbones, 4-arm backbones, 6-arm backbones, and 8-arm backbones. A multi-arm backbone that is particularly useful for the invention is the 4-arm PEG backbone, further described herein. In some cases, a reactive end group is attached to one end of the polymeric backbone.

The term “linker” as used herein refers to a structure of repeating units which are used to connect a photolabile group to a polymeric backbone through a chemical reaction. Linkers are known in the art and include such groups as alkyl chains which may be optionally substituted with heteroatoms such as oxygen, carbonyl groups, aldehyde groups, ketone groups, halogens, nitro groups, amide groups, and combinations thereof, as well as any group that does not prevent the desired reaction from occurring. In specific cases, linkers include “peptide linkers” and “PEG linkers.” The peptide linker consists of one or more amino acids, such as a peptide, oligopeptide or protein. The PEG linker is comprised of repeating units of the poly(ethylene glycol) moiety, —(CH₂—CH₂—O)ₙ—, where n is about 1 to about 200. In some cases, a reactive end group is attached to one end of the linker.

The term “amino acid” as used herein means an organic compound containing both a basic amino group and an acidic carboxyl group. Included within this term are natural amino acids (e.g., L-amino acids), modified and unusual amino acids (e.g., D-amino acids), as well as amino acids which are known to occur biologically in free or combined form but usually do not occur in proteins. Included within this term are modified and unusual amino acids, such as those disclosed in, for example, Roberts and Vellaccio (1983) The Peptides, 5: 342-429, the teaching of which is hereby incorporated by reference. Natural protein occurring amino acids include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, omithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-Cbz-protected amino acid, 2,4-diaminobutyric acid, homoarginine, N-methyl-arginine, norleucine, N-methylaminobutyric acid, naphthylalanine, phenylglycine, beta-phenylproline, tert-leucine, 4-aminocyclohexylalanine, N-methyl-norleucine, norvaline, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

The term “non-natural amino acid” is used to refer to an amino acid which does not exist on its own in nature, but rather, has been synthesized or created by man. Examples of non-natural amino acids include iodinated tyrosine, methylated tyrosine, glycosylated serine, glycosylated threonine, azetidine-2-carboxylic acid, 3,4-dehydroproline, perthiaproline, canavanine, ethionine, norleucine, selenomethionine, animohexanoic acid, telluromethionine, homoallylglycine, and homopropargylglycine. D-amino acids are also examples of non-natural amino acids.

The term “peptide bond” means a covalent amide linkage formed by loss of a molecule of water between the carboxyl group of one amino acid and the amino group of a second amino acid.

The term “peptide” refers to an organic compound comprising a chain of two or more amino acids covalently joined by peptide bonds. Peptides can be referred to with respect to the number of constituent amino acids, i.e., a dipeptide contains two amino acid residues, a tripeptide contains three, etc.

“N-terminus” refers to the first amino acid residue in a peptide chain or peptide linker. The N-terminal residue contains a free α-amino group.

“C-terminus” refers to the last amino acid residue in a peptide chain or peptide linker. The C-terminal residue contains a free carboxylate group.

As used herein, “therapeutic agent” includes those groups that cause a measurable physiological response in a mammal. The mammal may be human or non-human. Therapeutic agents are known in the art. All categories and specific therapeutic agents are intended to be included in this disclosure, even if not specifically mentioned. Therapeutic agents include, but are not limited to, enzymes, antibiotics, anesthetics, antibodies, growth factors, proteins, hormones, anti-inflammatories, analgesics, cardiac agents, vaccines and psychotropics.

As used herein, “caged groups” include those groups which may be activated upon photodegradation to elicit a fluorescent and/or chromogenic response, or a response that is detectable by other conventional analytical techniques. Caged groups can be attached to the photodegradable group, the end group, the backbone, or any other portion of the macromer. In one embodiment, caged groups are activated (have a different fluorescence or absorbance than when caged) upon photocleavage. This allows tracking of the progress of the photodegradation reaction. Fluorescein, bromohydroxycoumarin, fluorescent dyes and groups known in the art to be susceptible to two-photon photolysis are some useful caged groups, although there are other useful caged groups that are known in the art and that are intended to be included in this disclosure.

### II. Embodiments

### Photolabile Groups

The present invention provides a photodegradable composition having one or more photolabile nitrobenzo groups of formula I:

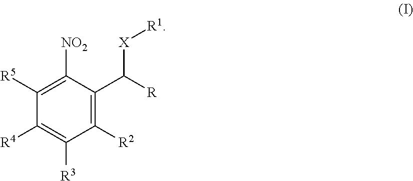

in which the one or more photolabile nitrobenzo groups comprises a polymeric backbone structure and a linker through which the backbone structure is attached to the photolabile nitrobenzo group;
X can be O, NH, or S;
R can be a hydrogen, a straight-chain or branched C₁-C₁₀ alkyl, aryl, alkoxy, aryloxy, or a carboxy group in which one or more carbon atoms can be independently optionally substituted with one or more heteroatoms, and one or more hydrogen atoms can be independently optionally substituted with hydroxyl, halogen, an oxygen atom, or backbone structure having at least one reactive end group, where the backbone structure can be appended through a peptide linker or a PEG linker, where the linker can have at least one reactive end group;
at least one of R¹, R², R³ R⁴, or R⁵ can be a backbone structure having at least one reactive end group, where the backbone structure can be appended through a peptide linker or a PEG linker, where the linker can have at least one reactive end group;
R¹, R², R³, R⁴, or R⁵ can each be independently selected from the group consisting of hydrogen; straight chain, branched or cyclic C₁-C₂₀ alkyl, alkenyl, alkynyl groups in which one or more of the carbon atoms are optionally substituted with non-hydrogen substituents and wherein one or more C, CH or CH₂ moiety can be replaced with an oxygen atom, a nitrogen atom, an —NR′ group, a —CO—R′ group, a S atom, or a reactive end group;
one or more R¹, R², R³ R⁴, or R⁵ can be optionally substituted with one or more substituents selected from halogen; nitro; cyano; isocyano; thiocyano; isothiocyano; azide; —SO₂; —OSO₃H; one or more optionally substituted straight-chain, branched or cyclic alkyl, alkenyl or alkynyl groups; OR′; —CO—OR′; —O—CO—R′; —N(R′)₂; —CO—N(R′)₂; —NR′—CO—OR′; —SR′; —SOR′; —SO₂—R′; —SO₃R′; —SO₂N(R′)₂; —P(R′)₂; —OPO₃(R′)₂; and —Si(R′)₃, wherein each R′, independent of other R′ in the substituent group can be a hydrogen, an optionally substituted straight-chain, branched or cyclic alkyl, alkenyl or alkynyl group wherein one or more C, CH or CH₂ groups therein can be replaced with an O atom, N atom, S atom or —NH group; an optionally substituted aromatic group, two or more R groups can be linked together to form a ring which may contain one or more of the same or different heteroatoms; and
R′ can in turn be optionally substituted with one or more groups selected from the group consisting of halogens, reactive end groups, nitro groups; cyano groups; isocyano groups; thiocyano groups; isothiocyano groups; azide groups; —SO₂ groups; —OSO₃H groups; straight-chain, branched or cyclic alkyl, alkenyl or alkynyl groups; halogenated alkyl groups; hydroxyl groups; alkoxy groups; carboxylic acid and carboxylic ester groups; amine groups; carbamate groups, thiol groups, thioether and thioester groups; sulfoxide groups, sulfone groups; sulfide groups; sulfate and sulfate ester groups; sulfonate and sulfonate ester groups; sulfonamide groups, sulfonate ester groups; phosphine groups; phosphate and phosphate ester groups; phosphonate and phosphonate ester groups; and alkyl-substituted silyl groups.

In some embodiments of the invention, R can be a hydrogen or a straight-chain or branched C₁-C₅ alkyl. In some other embodiments, R¹ can be a —CO—R′ group and R′ is a substituted alkyl having a reactive end group. The reactive end group can be an azide group or an alkynyl group. In further embodiments of the invention, R² and R⁵ can each be hydrogen. In another embodiment, R³ can be alkoxy or substituted alkoxy, where the alkoxy can be substituted with carboxylic acid. In some embodiments, R⁴ can be alkoxy or substituted alkoxy, where the alkoxy can be substituted with carboxylic acid.

In one aspect, the one or more photolabile nitrobenzo groups can have formula I-A:

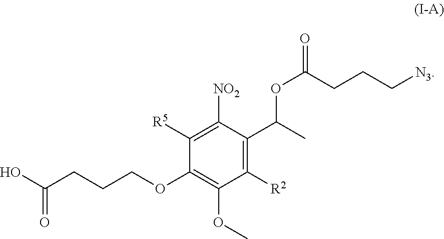

In another aspect, the one or more photolabile nitrobenzo groups can have formula I-B:

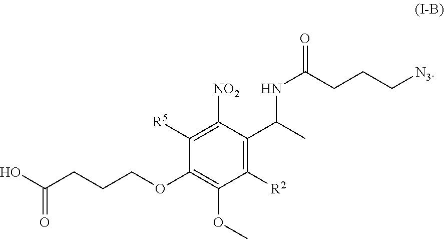

In another aspect, the one or more photolabile nitrobenzo groups can have formula I-C:

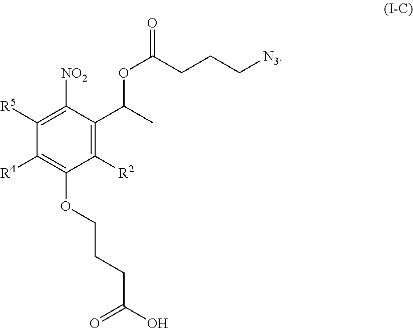

In another aspect, the one or more photolabile nitrobenzo groups can have formula I-D:

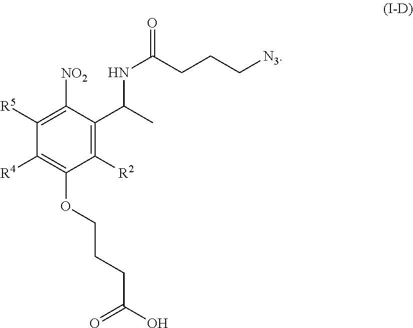

The present invention provides a photodegradable composition having one or more coumarin-azide cinnamic photorelease groups of formula II:

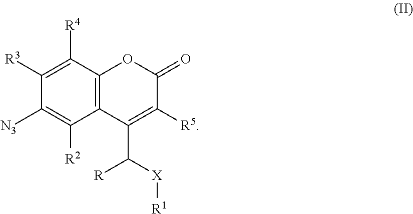

in which the one or more coumarin-azide cinnamic photorelease groups comprises a polymeric backbone structure and a linker through which the backbone structure is attached to the coumarin-azide cinnamic photorelease group;
X can be O, NH, or S;
R can be a hydrogen, a straight-chain or branched C₁-C₁₀ alkyl, aryl, alkoxy, aryloxy, or a carboxy group in which one or more carbon atoms can be independently optionally substituted with one or more heteroatoms, and one or more hydrogen atoms can be independently optionally substituted with hydroxyl, halogen, an oxygen atom, or backbone structure having at least one reactive end group, where the backbone structure can be appended through a peptide linker or a PEG linker, where the linker can have at least one reactive end group;
at least one of R¹, R², R³ R⁴, or R⁵ can be a backbone structure having at least one reactive end group, where the backbone structure can be appended through a peptide linker or a PEG linker, where the linker can have at least one reactive end group;
R¹, R², R³, R⁴, or R⁵ can each be independently selected from the group consisting of hydrogen; straight chain, branched or cyclic C₁-C₂₀ alkyl, alkenyl, alkynyl groups in which one or more of the carbon atoms are optionally substituted with non-hydrogen substituents and wherein one or more C, CH or CH₂ moiety can be replaced with an oxygen atom, a nitrogen atom, an —NR′ group, a —CO—R′ group, a S atom, or a reactive end group;
one or more R¹, R², R³ R⁴, or R⁵ can be optionally substituted with one or more substituents selected from halogen; nitro; cyano; isocyano; thiocyano; isothiocyano; azide; —SO₂; —OSO₃H; one or more optionally substituted straight-chain, branched or cyclic alkyl, alkenyl or alkynyl groups; OR′; —CO—OR′; —O—CO—R′; —N(R′)₂; —CO—N(R′)₂; —NR′—CO—OR′; —SR′; —SOR′; —SO₂—R′; —SO₃R′; —SO₂N(R′)₂; —P(R′)₂; —OPO₃(R′)₂; and —Si(R′)₃, wherein each R′, independent of other R′ in the substituent group can be a hydrogen, an optionally substituted straight-chain, branched or cyclic alkyl, alkenyl or alkynyl group wherein one or more C, CH or CH₂ groups therein can be replaced with an O atom, N atom, S atom or —NH group; an optionally substituted aromatic group, two or more R′ groups can be linked together to form a ring which may contain one or more of the same or different heteroatoms; and
R′ can in turn be optionally substituted with one or more groups selected from the group consisting of halogens, reactive end groups, nitro groups; cyano groups; isocyano groups; thiocyano groups; isothiocyano groups; azide groups; —SO₂ groups; —OSO₃H groups; straight-chain, branched or cyclic alkyl, alkenyl or alkynyl groups; halogenated alkyl groups; hydroxyl groups; alkoxy groups; carboxylic acid and carboxylic ester groups; amine groups; carbamate groups, thiol groups, thioether and thioester groups; sulfoxide groups, sulfone groups; sulfide groups; sulfate and sulfate ester groups; sulfonate and sulfonate ester groups; sulfonamide groups, sulfonate ester groups; phosphine groups; phosphate and phosphate ester groups; phosphonate and phosphonate ester groups; and alkyl-substituted silyl groups.

In some embodiments of the invention, R can be a hydrogen or a straight-chain or branched C₁-C₅ alkyl. In some other embodiments, R¹ can be a —CO—R′ group and R′ is a substituted alkyl having a reactive end group. The reactive end group can be an azide group or an alkynyl group. In further embodiments of the invention, R² and R⁵ can each be hydrogen. In another embodiment, R³ can be alkoxy or substituted alkoxy, where the alkoxy can be substituted with carboxylic acid. In some embodiments, R⁴ can be alkoxy or substituted alkoxy, where the alkoxy can be substituted with carboxylic acid.

In one aspect, the one or more coumarin-azide cinnamic photorelease groups can have formula II-A:

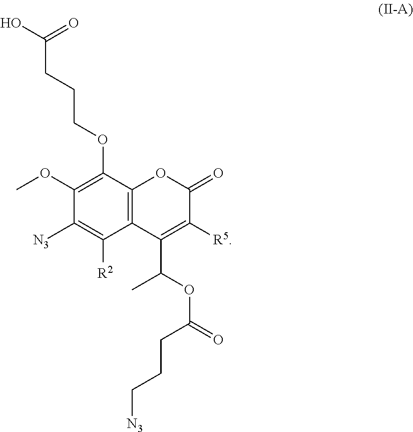

In another aspect, the one or more coumarin-azide cinnamic photorelease groups can have formula II-B:

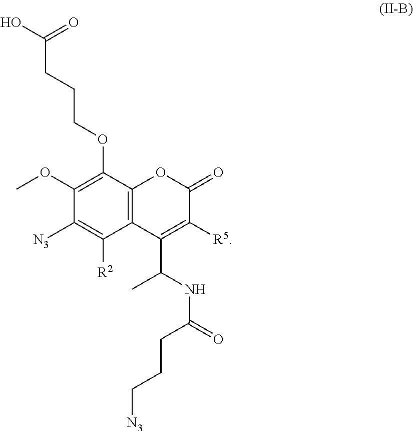

In another aspect, the one or more coumarin-azide cinnamic photorelease groups can have formula II-C:

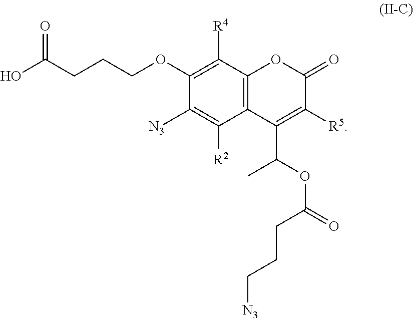

In another aspect, the one or more coumarin-azide cinnamic photorelease groups can have formula II-D:

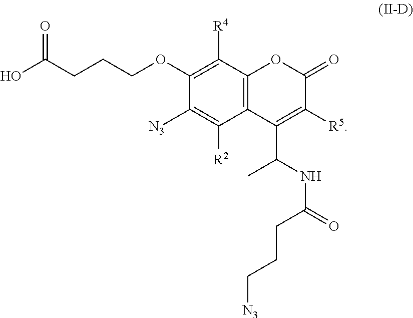

The present invention provides a photodegradable composition having one or more coumarin cinnamic photorelease groups of formula III:

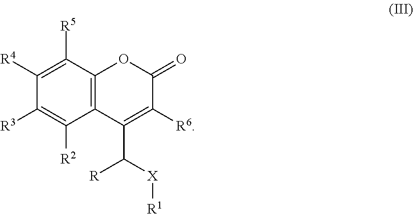

in which the one or more coumarin cinnamic photorelease groups comprises a polymeric backbone structure and a linker through which the backbone structure is attached to the coumarin cinnamic photorelease group;
X can be O, NH, or S
R can be a hydrogen, a straight-chain or branched C₁-C₁₀ alkyl, aryl, alkoxy, aryloxy, or a carboxy group in which one or more carbon atoms can be independently optionally substituted with one or more heteroatoms, and one or more hydrogen atoms can be independently optionally substituted with hydroxyl, halogen, an oxygen atom, or backbone structure having at least one reactive end group, where the backbone structure can be appended through a peptide linker or a PEG linker, where the linker can have at least one reactive end group;
at least one of R¹, R², R³ R⁴, R⁵, or R⁶ can be a backbone structure having at least one reactive end group, where the backbone structure can be appended through a peptide linker or a PEG linker, where the linker can have at least one reactive end group;
R¹, R², R³ R⁴, R⁵, or R⁶ can each be independently selected from the group consisting of hydrogen; straight chain, branched or cyclic C₁-C₂₀ alkyl, alkenyl, alkynyl groups in which one or more of the carbon atoms are optionally substituted with non-hydrogen substituents and wherein one or more C, CH or CH₂ moiety can be replaced with an oxygen atom, a nitrogen atom, an —NR′ group, a —CO—R′ group, a S atom, or a reactive end group;
one or more R¹, R², R³ R⁴, R⁵, or R⁶ can be optionally substituted with one or more substituents selected from halogen; nitro; cyano; isocyano; thiocyano; isothiocyano; azide; —SO₂; —OSO₃H; one or more optionally substituted straight-chain, branched or cyclic alkyl, alkenyl or alkynyl groups; OR′; —CO—OR′; —O—CO—R′; —N(R′)₂; —CO—N(R′)₂; —NR′—CO—OR′; —SR′; —SOR′; —SO₂—R′; —SO₃R′; —SO₂N(R′)₂; —P(R′)₂; —OPO₃(R′)₂; and —Si(R′)₃, wherein each R′, independent of other R′ in the substituent group can be a hydrogen, an optionally substituted straight-chain, branched or cyclic alkyl, alkenyl or alkynyl group wherein one or more C, CH or CH₂ groups therein can be replaced with an O atom, N atom, S atom or —NH group; an optionally substituted aromatic group, two or more R′ groups can be linked together to form a ring which may contain one or more of the same or different heteroatoms; and
R′ can in turn be optionally substituted with one or more groups selected from the group consisting of halogens, reactive end groups, nitro groups; cyano groups; isocyano groups; thiocyano groups; isothiocyano groups; azide groups; —SO₂ groups; —OSO₃H groups; straight-chain, branched or cyclic alkyl, alkenyl or alkynyl groups; halogenated alkyl groups; hydroxyl groups; alkoxy groups; carboxylic acid and carboxylic ester groups; amine groups; carbamate groups, thiol groups, thioether and thioester groups; sulfoxide groups, sulfone groups; sulfide groups; sulfate and sulfate ester groups; sulfonate and sulfonate ester groups; sulfonamide groups, sulfonate ester groups; phosphine groups; phosphate and phosphate ester groups; phosphonate and phosphonate ester groups; and alkyl-substituted silyl groups.

In some embodiments of the invention, R can be a hydrogen or a straight-chain or branched C₁-C₅ alkyl. In some other embodiments, R¹ can be a —CO—R′ group and R′ is a substituted alkyl having a reactive end group. The reactive end group can be an azide group or an alkynyl group. In further embodiments of the invention, R², R⁵ and R⁶ can each be hydrogen. In another embodiment, R³ can be alkoxy or substituted alkoxy, where the alkoxy can be substituted with carboxylic acid. In some embodiments, R⁴ can be alkoxy or substituted alkoxy, where the alkoxy can be substituted with carboxylic acid.

In one aspect, the one or more coumarin cinnamic photorelease groups can have formula III-A:

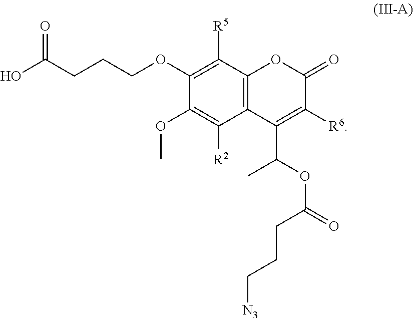

In another aspect, the one or more coumarin cinnamic photorelease groups can have formula III-B:

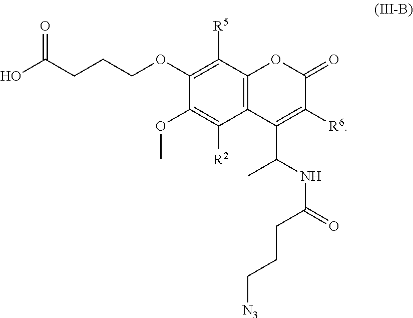

In another aspect, the one or more coumarin cinnamic photorelease groups can have formula III-C:

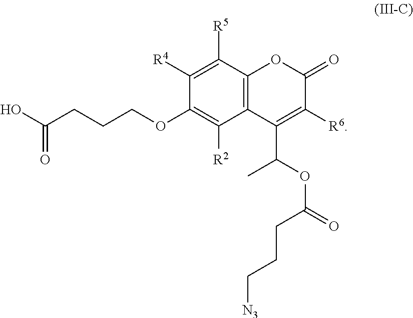

In another aspect, the one or more coumarin cinnamic photorelease groups can have formula III-D:

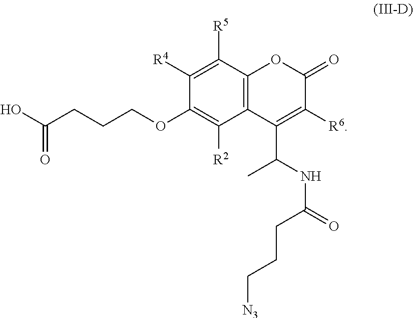

### Reactive End Groups

Any suitable reactive end group is useful in the photodegradable compositions of the present invention. The reactive end groups can be groups that are polymerizable by cationic, anionic, coordination, free-radical, condensation, and/or click reactions. In some embodiments of the invention, the one or more reactive end groups can be attached to any or all of the photolabile groups described herein, to any or all of polymeric backbones described herein, or to any or all of the linkers described herein.

In some embodiments of the invention, the photolabile groups of formulae I(A-D)-III(A-D) can have a structure in which X is O and R¹ is a —CO-alkyl substituted with a reactive end group:

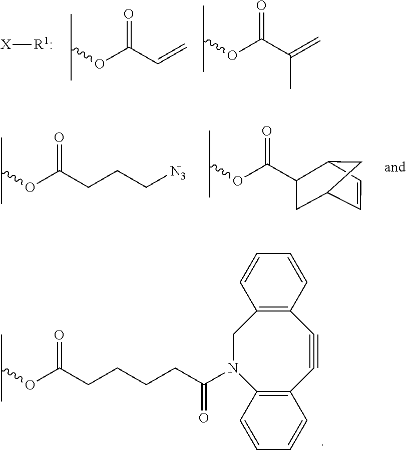

In some embodiments of the invention, the photolabile groups of formulae I(A-D)-III(A-D) can have a structure in which X is either O or NH and R¹ is a —CO-alkyl substituted with a reactive end group, which is an azide. For example, the photolabile nitrobenzo groups of formulae I-A, I-B, I-C, and I-D contain a reactive end group substituted on the R¹ position, in which the reactive end group is an azide attached to the ester alkyl end of a —CO—(CH₂)₃— group. In a further example, coumarin-azide cinnamic photorelease groups of formulae II-A, II-B, II-C, and II-D contain a an azide on the coumarin ring in addition to an additional reactive end group (azide) attached to the ester alkyl end of a —CO—(CH₂)₃— group. In yet a further example, the coumarin cinnamic photorelease groups of formulae III-A, III-B, III-C, and III-D contain a single reactive end group substituted on the R¹ position, in which the reactive end group is an azide attached to the ester alkyl end of a —CO—(CH₂)₃— group.

Further embodiments of the invention in which the polymeric backbone structures and the linkers contain a reactive end group are described below.

### Polymeric Backbone Structures

Any suitable polymeric backbone structure is useful in the photodegradable compositions of the present invention. The polymeric backbone structure can be attached to any one of the photolabile groups of formulae I(A-D)-III(A-D) through a linker. The polymeric backbone structure of the present invention can be comprised of one or more repeating units that may be the same or different. In some embodiments of the invention, the linker can contain a reactive end group. Representative backbone structures, which are in no way meant to be limiting, are included in Table 1 below.

TABLE 1
Polymeric backbones of the photodegradable composition^{a}

| | |
|---|---|
| poly(ethylene glycol) (PEG) | 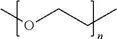 |
| poly(ethylene oxide) | 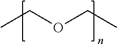 |
| poly(vinyl alcohol) | 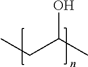 |
| poly(vinylpyrrolidone) | 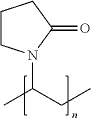 |
| poly(styrene) | 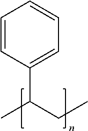 |
| poly(acrylate) | 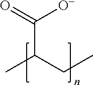 |
| poly(methacrylates) | 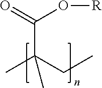 |
| poly(vinylethers) | 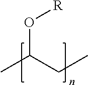 |
| poly(urethanes) | 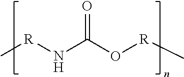 |
| polypropylene | 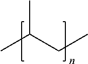 |
| polyester | 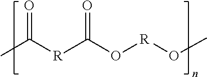 |
| polyethylene | 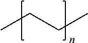 |
| “polyethoxy bis- propanamide” | 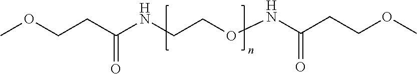 |

^{a}R is as defined herein; n is 1-200.

In certain aspects of the invention, the polymeric backbone structure can be a “multi-arm” backbone structure. A “multi-arm” backbone structure can be a 3-arm, 4-arm, 6-arm, 8-arm, etc., backbone structure. In some embodiments, the multi-arm backbone structure can be a 4-arm backbone structure having one or more repeating PEG units. The multi-arm-PEG backbone structure can be functionalized with a reactive end group, including, but not limited to, acrylate, methacrylate, styrene, allyl ether, vinyl ether, isocyanate, cyanoacrylate, norbomylene, azide, dibenzocyclooctene (DBCO), triazide, phosphazine, imine, oxazoline, propylene sulfide, or thiol groups. In some embodiments, multi-arm-PEG backbone structure can be functionalized with a reactive end group DBCO or thiol. In certain embodiments, multi-arm-PEG backbone structure can be a 4-arm PEG backbone structure functionalized with reactive end group DBCO or thiol. In other embodiments, the functionalized 4-arm-PEG backbone structure can be 4-arm poly(ethylene glycol) tetradibenzocyclooctyne (i.e., 4-arm-PEG-DBCO) or poly(ethylene glycol) tetrathiol (i.e., PEG₄SH).

In certain aspects, the photodegradable composition can have a 4-arm-PEG-DBCO polymeric backbone. In one aspect, the 4-arm-PEG-DBCO polymeric backbone has the following formula IV:

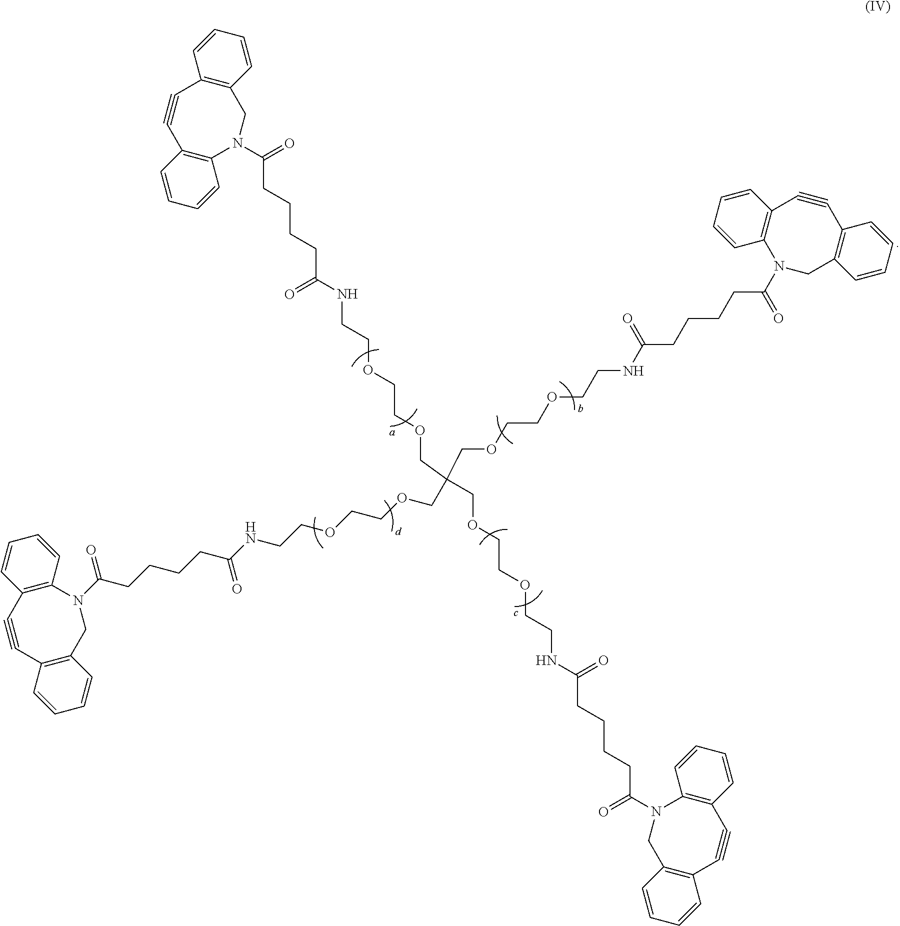

in which subscripts a, b, c, and d are each independently selected from 1 to 200, such as 10, 20, 30, 50, 80, 100, 125, 130, 140, 150, 160, 170, 180, 190, or 200.

### Linkers

Any suitable linker is useful in the photodegradable compositions of the present invention. The linker can be used to connect any or all of the photolabile groups of formulae I(A-D)-III(A-D) to any or all of polymeric backbones described herein. The linker of the present invention can have one or more repeating unit repeating units of amino acid monomers joined by peptide bonds (i.e. peptide linker). In other embodiments, the linker can have one or more repeating units of the poly(ethylene glycol) moiety, —(CH₂—CH₂—O)ₙ—, where n can be about 1 to about 200 (i.e. PEG linker). In some embodiments, n can be 10, 20, 30, 50, 80, 100, 125, 130, 140, 150, 160, 170, 180, 190, or 200. In other embodiments of the invention, the linker can be functionalized with a reactive end group.

In some embodiments of the invention, the peptide linker can be an amino acid polymer having a plurality of peptide bonds, and is not limited by the number of amino acid residues included in the peptide chain, with the term typically referring to one having a relatively small molecular weight with about 5, 10, 15, 20, 25, 30, 35, 40, 45, or 50 amino acid residues. In certain other instances, the peptide linker has about 2-50 amino acids such as 1, 2, 3, 4, 5, 6, 7, 8, 9, 10, 11, 12, 13, 14, 15, 16, 17, 18, 19, 20, 21, 22, 23, 24, 26, 26, 28 29, 30, 40, 50 or more amino acids.

The amino acids disclosed herein are indicated by single-letter designations (in sequence listings) in accordance with the nomenclature for amino acids set forth in the IUPAC-IUB guidelines. In the present description, the term “synthetic peptide” refers to a peptide fragment that is manufactured by artificial chemical synthesis or biosynthesis (i.e. genetic engineering-based production).

In certain aspects, the amino acid monomers making the peptide linker comprise members selected from the group consisting of A, C, D, E, F, G, H, I, K, L, M, N, P, Q, R, S, T, V, W, and combinations thereof. Hydroxyl-containing, and charged amino acids are preferred. Each letter represents an amino acid. In certain embodiments, the peptide linker can have the following amino acid chain: -RGGRK-, in which the N terminus comprises a reactive end group and the C terminus comprises an amine.

In certain aspects, the photodegradable composition can have the -RGGRK-peptide linker with the following formula V:

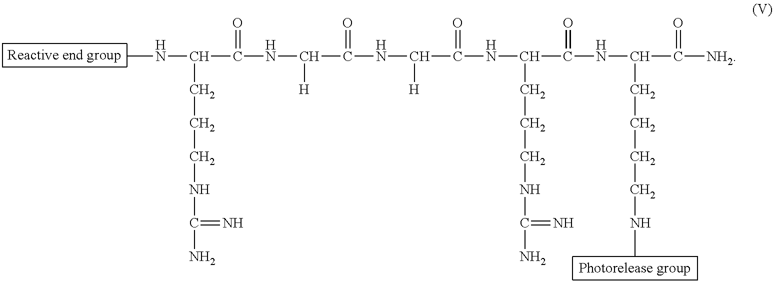 [SMILES: CNCCCCC(NC(=O)C(CCCNC(=N)N)NC(=O)CNC(=O)CNC(=O)C(CCCNC(=N)N)NC)C(N)=O]

In some embodiments, the photodegradable composition can have a peptide linker of formula V, where the photorelease group is a photolabile nitrobenzo group of formula I-A, and the reactive end group is an azide attached through a —CO—(CH₂)₃— group on the linker, having the following formula V-A:

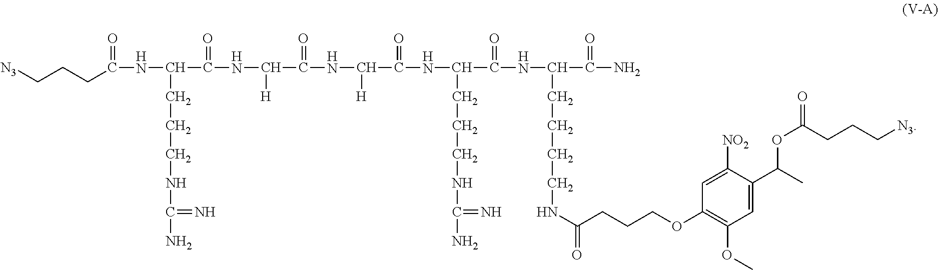 [SMILES: COc1cc(C(C)OC(=O)CCCN=[N+]=[N-])c([N+](=O)[O-])cc1OCCCC(=O)NCCCCC(NC(=O)C(CCCNC(=N)N)NC(=O)CNC(=O)CNC(=O)C(CCCNC(=N)N)NC(=O)CCCN=[N+]=[N-])C(N)=O]

In some embodiments, the photodegradable composition can have a peptide linker of formula V, where the photorelease group is a coumarin-azide cinnamic photorelease group of formula II-A, and the reactive end group is an azide attached through a —CO—(CH₂)₃— group on the linker, having the following formula V-B:

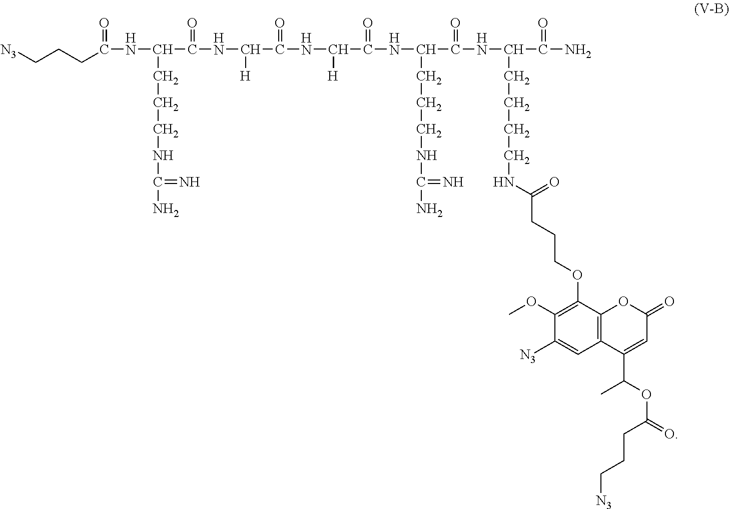 [SMILES: COc1c(N=[N+]=[N-])cc2c(C(C)OC(=O)CCCN=[N+]=[N-])cc(=O)oc2c1OCCCC(=O)NCCCCC(NC(=O)C(CCCNC(=N)N)NC(=O)CNC(=O)CNC(=O)C(CCCNC(=N)N)NC(=O)CCCN=[N+]=[N-])C(N)=O]

In some embodiments, the photodegradable composition can have a peptide linker of formula V, where the photorelease group is a coumarin cinnamic photorelease group of formula III-A, and the reactive end group is an azide attached through a —CO—(CH₂)₃— group on the linker, having the following formula V-C:

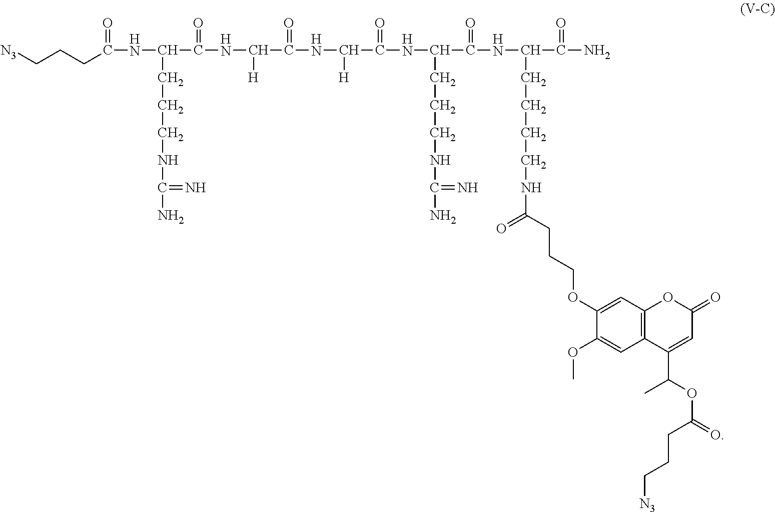 [SMILES: COc1cc2c(C(C)OC(=O)CCCN=[N+]=[N-])cc(=O)oc2cc1OCCCC(=O)NCCCCC(NC(=O)C(CCCNC(=N)N)NC(=O)CNC(=O)CNC(=O)C(CCCNC(=N)N)NC(=O)CCCN=[N+]=[N-])C(N)=O]

In some embodiments, the PEG linker can be a poly(ethylene glycol) polymer having a plurality of —(CH₂—CH₂—O)ₙ— units. In certain aspects of the invention, the PEG linker contains a reactive end group, and has the following formula VI:

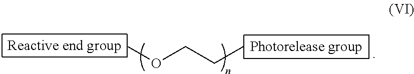

The n indicates the number of PEG units present in the PEG linker and can be about 1 to about 200. In some embodiments, the PEG linker can have about 1, 5, 20, 40, 60, 80, 100, 120, 140, 160, 180, or 200 PEG units in the PEG chain. In other embodiments, the PEG linker can have about 1 to about 200 PEG units, or about 5 to about 180, or about 20 to about 160, or about 40 to about 140, or about 60 to about 120, or about 80 to about 100 PEG units in the PEG chain. In certain embodiments, the PEG linker can have about 5 to about 50 PEG units in the PEG chain. In other embodiments, the PEG linker can have about 24, 34 or 44 PEG units in the PEG chain. In some other embodiments, the PEG linker can have about 34 PEG units in the PEG chain.

The PEG linker can have a molecular weight of about from about 1 to about 45 kDa. In some embodiments, the PEG linker can have a molecular weight of about 1, 1.5, 3, 3.5, 5, 10, 15, 20, 25, 30, 35, or about 40 kDa. In other embodiments, the PEG linker can have a molecular weight of about 1 to about 40 kDa, or about 1.5 to about 35, or about 3 to about 30, or about 3.5 to about 25, or about 5 to about 20, or about 10 to about 15 kDa. In certain embodiments, the PEG linker can have a molecular weight of about 1 to about 5 kDa. In other embodiments, the PEG linker can have a molecular weight of about 1.5.

In certain aspects of the invention, the photodegradable composition can have the PEG linker with the following formula VII:

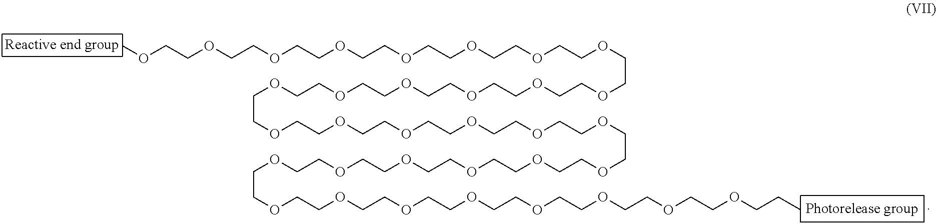 [SMILES: CCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOC]

In some embodiments of the invention, the photodegradable composition can have a PEG linker of formula VII, where the photorelease group is a photolabile nitrobenzo group of formula I-A, and the reactive end group is an azide attached through a —CO—(CH₂)₃— group on the linker, having the following formula VII-A:

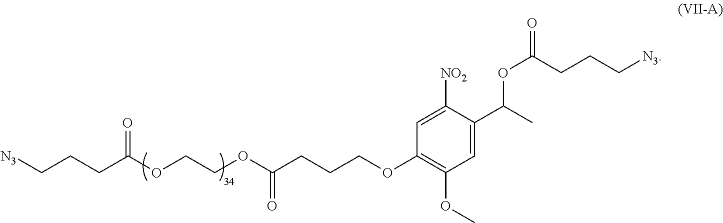

In some embodiments of the invention, the photodegradable composition can have a PEG linker of formula VII, where the photorelease group is a coumarin-azide cinnamic photorelease group of formula II-A, and the reactive end group is an azide attached through a —CO—(CH₂)₃— group on the linker, having the following formula VII-B:

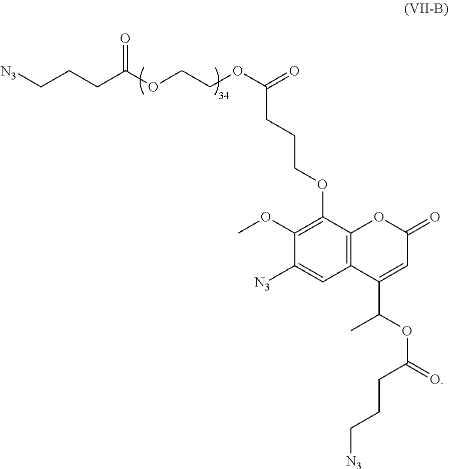

In some embodiments of the invention, the photodegradable composition can have a PEG linker of formula VII, where the photorelease group is a coumarin cinnamic photorelease group of formula III-A, and the reactive end group is an azide attached through a —CO—(CH₂)₃— group on the linker, having the following formula VII-C:

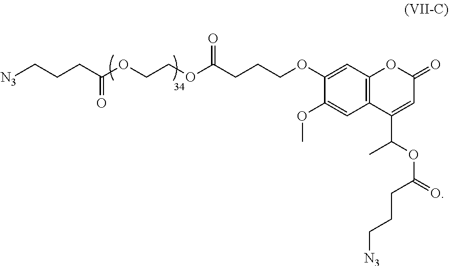

### Therapeutic Agents and Caged Groups

In certain embodiments of the invention, the photodegradable composition can be formed by reacting any one or more of the polymeric backbones described herein with any one or more of the photolabile groups described herein containing any one of the linkers described herein. Each of the polymeric backbones, photolabile groups, and linkers can be functionalized with at least one reactive end group. In some embodiments, the polymeric backbone can be a multi-arm polyethylene glycol PEG polymer functionalized with dibenzocyclooctyne (DBCO) reactive groups. In other embodiments, the photolabile group can be a photolabile group of formulae I-III. In some embodiments, the photolabile group can be a photolabile nitrobenzo group of formulae I-A, I-B, I-C, or I-D. In other embodiments, the photolabile group can be a coumarin-azide cinnamic photorelease group of formulae II-A, II-B, II-C, and II-D. In further embodiments, the photolabile group can be a coumarin cinnamic photorelease group of formulae III-A, III-B, III-C, and III-D. In other embodiments, the linker can be a peptide linker of formula V or a PEG linker of formula VI. The photodegradable composition can be formed via a click reaction between any of the reactive end groups on the polymeric backbone and the photolabile groups and linkers. In some aspects, the photodegradable composition is a step-growth network. In certain aspects, the photodegradable composition can be in the form of a hydrogel, a microparticle, a nanoparticle, or a thin film.

In a particular aspect of the invention, the photodegradable composition can be formed by reacting the at least one reactive end group of a polymeric backbone with the at least one reactive end group of the photolabile group and/or the at least one reactive end group of the linker. In one embodiment, the reaction (or reactions) between the at least one reactive end group of the polymeric backbone and the at least one reactive end group of the photolabile group and/or the at least one reactive end group of the linker is a click reaction. The click reaction between the at least one reactive end group of the polymeric backbone and the at least one reactive end group of the photolabile group and/or the at least one reactive end group of the linker produces the photodegradable composition of the invention, in which the photodegradable composition is in the form of a hydrogel. In certain embodiments, the polymeric backbone is the 4-arm polyethylene glycol PEG polymer functionalized with dibenzocyclooctyne (DBCO) reactive groups of formula IV. In further embodiments, the click reaction is performed using the polymeric backbone of formula IV and the compound of formula V-A, in which the peptide linker of formula V is attached to the photolabile nitrobenzo group of formula I-A, and the reactive end group is an azide attached through a —CO—(CH₂)₃— group on the linker. The click reaction between IV and V-A, shown in Scheme 1, results in the formation of the photodegradable composition of the invention in the form of a hydrogel.

The photodegradable composition in hydrogel network formation is accomplished by reacting the DBCO end groups of the polymeric backbone in a stoichiometric ratio with the azide groups on the linker-photolabile group species. The photodegradable hydrogel network composition is comprised of a series of fused-cycloocto-triazole ring systems formed upon the click reaction between an azide of formula V-A and an alkyne of formula IV. Scheme 2 shows the click reaction which occurs between the two reactive end groups on the components of V-A and IV, which form the photodegradable composition hydrogel network of the invention.

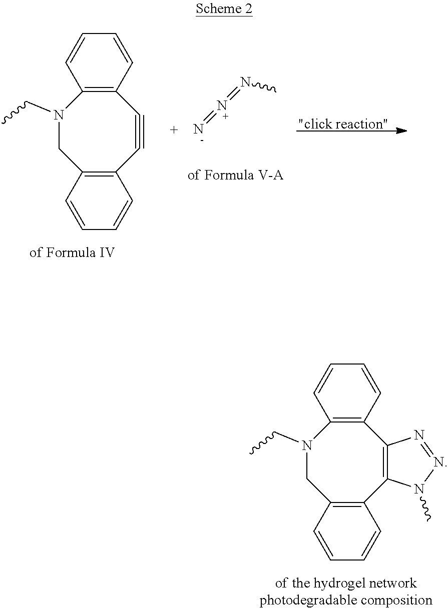

In certain aspects, the composition can contain an entrapped biomolecule, which biomolecule can be optionally releasable upon photodegradation of the composition. In some embodiments, the photodegradable composition containing an entrapped biomolecule can be photodegraded with light irradiation at a wavelength of from about 100 nm to about 1000 nm, thereby releasing the entrapped biomolecule. In some embodiments, the photodegradable composition containing an entrapped biomolecule can be photodegraded with light irradiation at a wavelength of from about 100 nm, or from about 200, 300, 400, 500, 600, 700, 800, 900 or 1000 nm, thereby releasing the entrapped biomolecule. In further embodiments, the photodegradable composition containing an entrapped biomolecule can be photodegraded with light irradiation at a wavelength of from about 100 nm to about 1000 nm, or from about 200 nm to about 900 nm, or from about 300 nm to about 800 nm, or from about 400 nm to about 700 nm, or from about 500 nm to about 600 nm, thereby releasing the entrapped biomolecule. In still other embodiments, the photodegradable composition containing an entrapped biomolecule can be photodegraded with light irradiation at a wavelength of from about 200 nm to about 500 nm, or about 390 nm to about 850 nm, or from about 400 nm to about 500 nm, thereby releasing the entrapped biomolecule. In further embodiments of the invention, the photodegradable composition containing an entrapped biomolecule can be photodegraded with light irradiation at a wavelength of about 365 nm, 390 nm, or 740 nm, thereby releasing the entrapped biomolecule.

In certain aspects, the photodegradable composition can contain an entrapped biomolecule, which can be a member selected from the group consisting of a protein, a peptide, an enzyme, an enzyme substrate, a vaccine, a hormone, an antibody, an antibody fragment, an antigen, a hapten, an avidin, a streptavidin, a carbohydrate, an oligosaccharide, a polysaccharide, a nucleic acid, a fragment of DNA, a fragment of RNA and a biological therapeutic. In some aspects, the enzyme can be a phosphatase, a ligase, or a galactosidase.

In certain aspects, the entrapped biomolecule can be a vaccine. In some embodiments, the vaccine can be a vaccine against a viral disease or a bacterial disease. In certain aspects, the viral caused disease can be selected from the group consisting of rabies, Hepatitis A, Hepatitis B, cervical cancer, genital warts, anogenital cancers, influenza, Japanese encephalitis, measles, mumps, rubella, poliomyelitis, rotaviral gastroenteritis, smallpox, chickenpox, shingles, and Yellow fever. In other aspects, the bacteria caused disease can be selected from the group consisting of Anthrax, Whooping cough, Tetanus, Diphtheria, Q fever, epiglottitis, meningitis, pneumonia, Tuberculosis, Meningococcal meningitis, Typhoid fever, Pneumococcal pneumonia and Cholera.

In some embodiments of the invention, the photodegradable composition can contain a stabilizer, such as a carbohydrate. In some embodiments, the stabilizer can be a trehalose or other sugars which are added to a hydrogel to control water of hydration. In addition to being added to the hydrogel formulation without covalent attachment, trehalose or other sugars can be functionalized as follows:

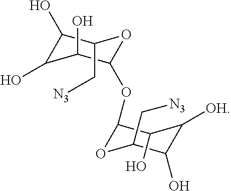 [SMILES: [N-]=[N+]=NCC1OC(OC2OC(CN=[N+]=[N-])C(O)C(O)C2O)C(O)C(O)C1O]

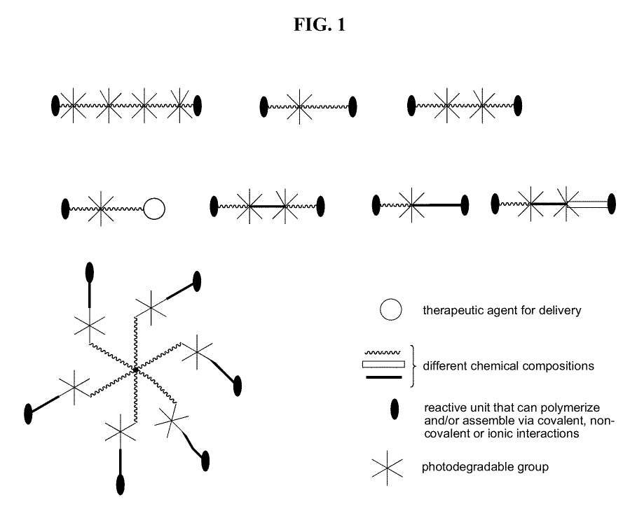

FIG. 1 shows some exemplary structures into which photodegradable groups can be incorporated according to the invention. Photodegradable groups can be incorporated into macromers, block copolymers, and linear and branched polymers, for example. They can be incorporated between a reactive end group, such as an olefin, and a therapeutic agent, for incorporation into a tissue scaffold to provide spatial and temporal control over the release of the agent. Photodegradable groups can be incorporated into linear structures and crosslinked structures to allow rapid and precise degradation of higher molecular weight materials. The macromers can form or be incorporated into networks via covalent, non-covalent and/or ionic interactions, as known in the art. These networks can be used for 3-D photolithography via single and multi-photon photolysis. Thin films of reacted macromers can be cast and then degraded for 2-D lithography. Incorporation of a chromagenic or fluorescent group (caged group) into the photodegradable linkage that is activated upon degradation allows for 2-D and 3-D imaging. The chromagenic or fluorescent group can be detected using any available technique.

The macromers can be amphiphilic, incorporating both hydrophobic and hydrophilic segments, or can be hydrophilic or hydrophobic. The macromers can be linear or branched, and can form linear, branched or crosslinked networks which are then photodegradable. These macromers can be incorporated or grafted onto surfaces to impart biocompatibility. The polymers and polymer networks formed from these macromers can, for example, undergo bulk degradation, surface degradation, gradient degradation and/or focused degradation that is spatially controllable. Multiple photodegradable groups which degrade at different wavelengths with or without a photosensitizer allows for multistage degradation, including surface and bulk patterning and spatial control over release of multiple groups. This can be used to control the timing and spatial release of therapeutics in different parts of the body, for example. The compositions of the invention can be combined with groups that undergo existing methods of degradation, such as hydrolysis or enzymatic degradation.

Incorporation of different photodegradable groups that photolyze at different wavelengths in one macromer or different macromers that are incorporated into a network allows a broad range of wavelengths to be used for photodegradation (such as those wavelengths ≥300 nm (including light around 365 nm) but preferably in the longwave ultra-violet to visible light region for biological applications (because shorter wavelengths such as 280 nm cause mutations, damage and/or cell death) and intensities, and allows for multi-stage degradation where the degradation is temporally controlled by the timing of the application of the appropriate cleaving photoradiation for each different photodegradable group, dual degradation of different photodegradable groups by the simultaneous application of different cleaving photoradiation for each photodegradable group and/or release of desired substances. The degradation of one photodegradable group at one wavelength can be simultaneous with or at a different time than the degradation of another photodegradable group at a different wavelength by application of the appropriate wavelength.

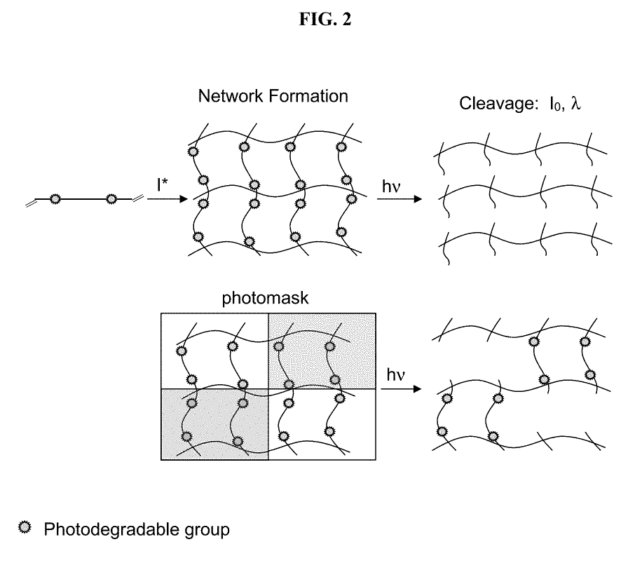

FIG. 2 shows one general description of the formation and cleavage of networks of macromers of the invention. A network is formed by the reaction of multiple photodegradable macromers with reactive end groups. Upon application of the appropriate wavelength and intensity of light, the photodegradable groups cleave (top of FIG. 2). Portions of the network can be masked using any material that the light does not penetrate, such as foil, a transparency film with printed black areas in a desired arrangement, or other masking materials known in the art, allowing the desired patterning of cleaved groups and uncleaved groups (bottom of FIG. 2). Sequential photodegradation of unmasked portions and masked portions then occurs by application of the appropriate wavelength.

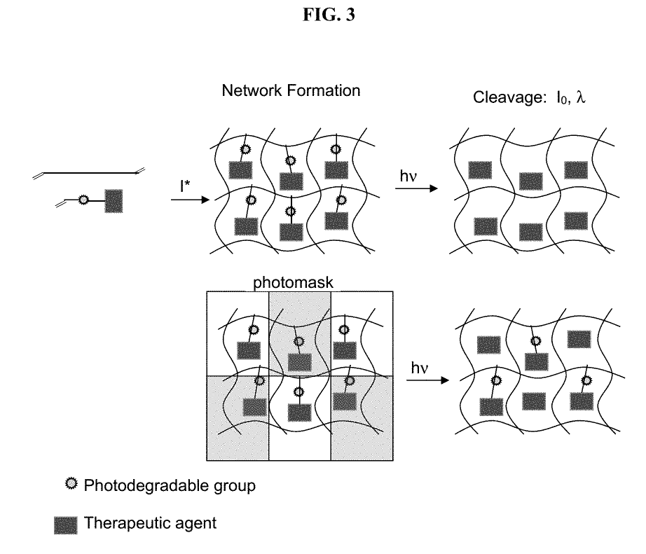

FIG. 3 shows one application of the invention using a therapeutic agent. As shown in FIG. 3, the network can be formed using different precursors, some having photodegradable groups with optional therapeutic agents which may be the same or different, and some not having photodegradable groups, allowing for the desired network composition. Upon application of light having the appropriate intensity and wavelength, the photodegradable groups cleave. Different photodegradable groups can be incorporated into the network to allow for degradation of different photodegradable groups with different light wavelengths. As shown in the bottom of FIG. 3, using a photomask, some of the photodegradable groups can be allowed to cleave upon the initial application of light and others can remain uncleaved. This allows the release of a portion of the therapeutic agent at one time and allows the release of a different portion of the therapeutic agent at a different time. Various combinations of therapeutic agents, caged groups, photodegradable groups, masks and other components can be used to provide the desired release profile by one of ordinary skill in the art without undue experimentation using the knowledge in the art and provided herein.

General Procedure for Controlled 2-D Degradation using a photomask: A photomask is contacted with the surface of the hydrogel. The gel can be degraded using a 5 cm collimated flood exposure source coupled to an optical mask alignment system (Optical Associates, Inc. San Jose, Calif.), which generates 50-70 mW cm⁻² of radiation (365 nm). An adjustable reaction chamber facilitates well-defined control over degradation. The spacing between the photomask and chamber bottom is controlled by micromanipulators coupled to a height sensor and the entire reaction chamber is integrated with the theta and lateral controls of the Mask aligner. Photomasks are made using emulsion films (Polychrome V; Kodak, Rochester, N.Y.) exposed with a high-resolution He—Ne red laser diode commercial plotter.

3-D Lithography may be accomplished using a series of photomasks with the mask alignment system described above, or through the use of a two-photon laser scanning microscope.

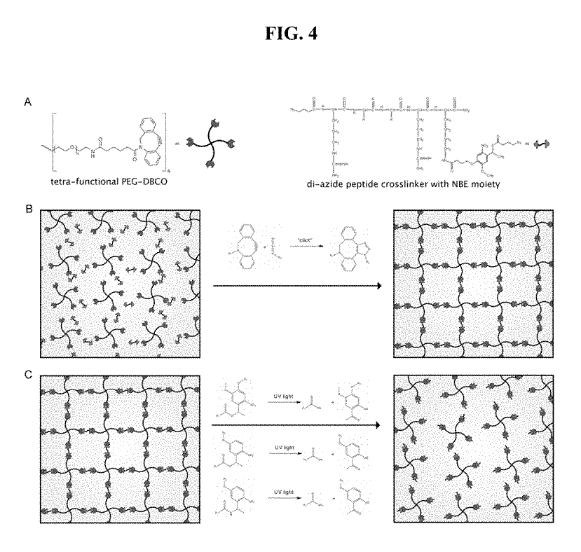

FIG. 4 shows an example of a photodegradable network formation and degradation, wherein a tetra-functional PEG-DBCO is reacted with a diazide peptide crosslinker with a nitrobenzyl ether (NBE) moiety. In FIG. 4B, tetra-functional PEG-DBCO and an azide-RGGRK-PL group-azide are conjugated via a strain promoted azide alkyne cycloaddition (SPAAC) “click” chemistry reaction. The reaction between the DBCO alkyne and the azide of the crosslinker to form a five membered covalently bonded ring occurs rapidly (<1 min) under physiologic conditions and is bio-orthogonal. FIG. 4C shows a photodegradation event with different formulations of an NBE moiety. The nitro group in this example is assisting with the degradation of the photo cleaved ester or amide. The three examples shown are illustrative and are not meant to limit the scope of the invention.

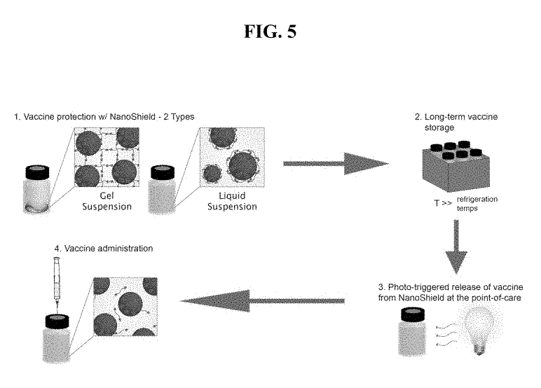

FIG. 5 illustrates that the invention can be used either as a dried gel film or liquid suspension in a nanoparticle or microparticle form to encapsulate and stabilize thermally-sensitive molecules such as vaccines. Thereafter, the temperature-sensitive payload can be transported and stored without the need for refrigeration. After long term storage, the payload is released from the invention after irradiation with the appropriate wavelengths of light and is thereby activated. Once activated, the payload can be directly administered as shown in the fourth panel of FIG. 5.

### III. Examples

### Example 1. Synthesis of 4-(4-(1-(4-azidobutanoyloxy)ethyl)-2-methoxy-5-nitrophenoxy)butanoic acid (PLazide, Azide-A)

4-[4-(-hydroxyethyl)-2-methoxy-5-nitrophenoxy)butyric acid (0.4 g, 1.3 mmol, purchased from Novabiochem, was dissolved in 12 mL dry dichloromethane (DCM) under argon. 4-Dimethylaminopyridine (DMAP) (8.0 mg, 0.065 mmol) and pyridine (0.108 mL, 1.34 mmol) was added while stirring. The reaction vessel was kept in an ice bath. 4-Azidobutanoic anhydride (1.44 g, 6 mmol) was added to the DCM solution and the reaction was stirred overnight. The crude mixture was washed with aq. NaHCO₃ (10 mL), 1 N HCl (10 mL), and brine (10 mL). The organic phase was dried over MgSO₄ and concentrated. This mixture was dissolved in 50:50 acetone/H₂O (140 mL) and stirred overnight. The acetone was removed by evaporation, and the product was extracted into DCM (3*25 mL). The organic layer was washed with 1 N HCl (50 mL), and brine (50 mL) and dried over MgSO₄, concentrated, and purified by flash chromatography (hexanes/ethylacetate).

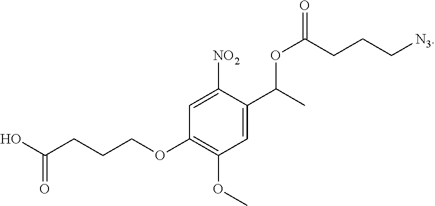 [SMILES: COc1cc(C(C)OC(=O)CCCN=[N+]=[N-])c([N+](=O)[O-])cc1OCCCC(=O)O]

The structure above, Azide-A, is one PLazide photodegradable ligand. The carboxylic acid can be attached to both PEG linkers of various lengths and free amines on peptides of various lengths and compositions.

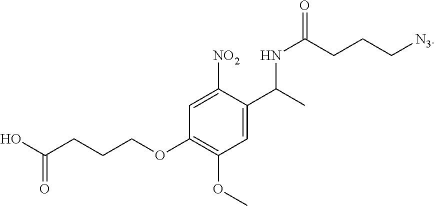 [SMILES: COc1cc(C(C)NC(=O)CCCN=[N+]=[N-])c([N+](=O)[O-])cc1OCCCC(=O)O]

The structure above, Azide-B, is one photodegradable ligand with an amide instead of ester (OMEamide).

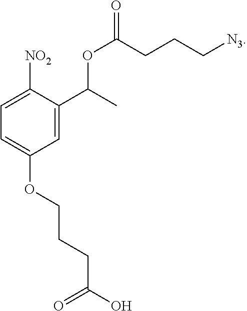 [SMILES: CC(OC(=O)CCCN=[N+]=[N-])c1cc(OCCCC(=O)O)ccc1[N+](=O)[O-]]

The structure above, Azide-C, is one photodegradable labile group.

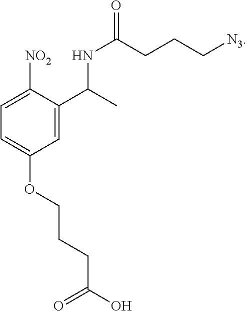 [SMILES: CC(NC(=O)CCCN=[N+]=[N-])c1cc(OCCCC(=O)O)ccc1[N+](=O)[O-]]

The structure above, Azide-D, is one amide version of photo labile ligand. The rate of photo degradation is Azide-4>Azide-3>Azide-2>Azide-1.

### Example 2. Synthesis of the Azide-RGGRK(PLazide)-NH2. (V-A)

For the synthesis of azide-functionalized photodegradable peptide crosslinker, 0.5 mmol of peptide H-RGGRK(dde)-NH₂ was synthesized (Protein Technologies Tribute peptide synthesizer) through Fmoc solid-phase methodology and HATU activation. 4-Azidobutanoic acid was coupled to the N-terminal amine with HATU, the 1-(4,4-dimethyl-2,6-dioxacyclohexylidene)ethyl (dde) group was removed with 2% hydrazine_monohydrate (Sigma) in DMF (3×10 min), and 4-(4-(1-(4-azidobutanoyloxy)ethyl)-2-methoxy-5-nitrophenoxy)butanoic acid (PLazide) was coupled to the 1-amino group of the C-terminal lysine. Resin was treated with trifluoroacetic acid/triisopropylsilane/water (95:2.5:2.5) for 2 h and precipitated in and washed (2×) with ice-cold diethyl ether. The crude peptide was purified using semi-preparative reversed-phase high-performance liquid chromatography (RP-HPLC) (Waters Delta Prep 4000) using a 70 min linear gradient (5-95% of acetonitrile and 0.1% trifluoroacetic acid) and lyophilized to give the product (Azide-RGGRK(PLazide)-NH₂) as a fluffy, yellow solid. Peptide purity was confirmed with analytical RP-HPLC and matrix-assisted laser desorption-ionization time-of-flight mass spectrometry (Applied Biosystems DE Voyage) using a-cyano-4-hydroxycinnamic acid matrix (Sigma): calculated ([MH]⁺1,060.1); observed ([MH]⁺1058).

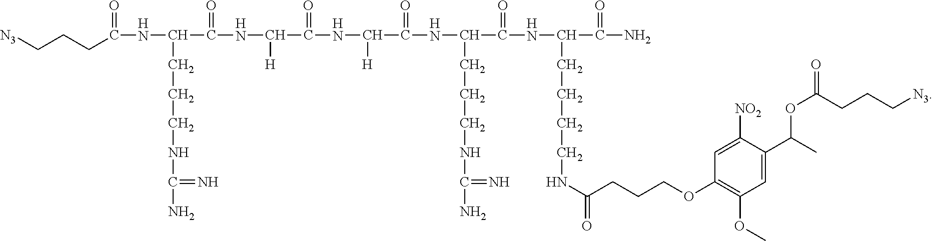 [SMILES: COc1cc(C(C)OC(=O)CCCN=[N+]=[N-])c([N+](=O)[O-])cc1OCCCC(=O)NCCCCC(NC(=O)C(CCCNC(=N)N)NC(=O)CNC(=O)CNC(=O)C(CCCNC(=N)N)NC(=O)CCCN=[N+]=[N-])C(N)=O]

The structure V-A above is an Azide-RGGRK(PLazide)-NH₂ peptide linker. A skilled artisan will appreciate that peptide linkers are not limited to this peptide configuration or length of linker. The amino acids can be altered or expanded. The two characteristic features are the terminal azide at both ends and the photolabile nitro-benzo group The peptide is synthesized on Protein Technologies Tribute peptide synthesizer through Fmoc solid-phase methodology and HATU activation and can be modified to include other amino acids, protected amino acids or functionalized amino acids to modify solubility and/or reactivity.

### Example 3. Synthesis of four-arm poly(ethylene glycol) (PEG) tetradibenzocyclooctvne (Mn≈20,000 Da) (4-arm-PEG-DBCO)

Dibenzocyclooctyne acid (DBCO-Acid) (100 mg, 0.33 mmol) and 1-[Bis(dimethylamino)methylene]-1H-1,2,3-triazolo[4,5-b]pyridinium 3-oxid hexafluorophosphate (HATU) (114.04 rag, 0.3 mmol) was dissolved in 1 mL N,N-dimethylformamide (DMF). A 4 arm PEG tetraamine (PEG) (Mₙ≈20,000 Da) (1.363 g, 0.068 mmol) was pre-dissolved in 3 mL DMF. N,N-Diisopropylethylamine (DIEA) (52.25 μL) was added to the DBCO-Acid/HATU solution and allowed to stir for 3 min, at which time the PEG solution was added. The PEG vial was washed with an additional 1 mL DMF. The final solution was stirred overnight at room temperature. The next day the solution was taken to dryness using reduced pressure rotary evaporator. Solid was dissolved in approx. 35 mL of DI H₂O and dialyzed for 3 days in SpectraPur 7 MWCO 3500 Da dialysis tubing. Water was changed every 12 hr. The resulting solution was frozen and lyophilized to yield the four-arm poly(ethylene glycol) tetradibenzocyclooctyne (Mₙ≈20,000-21,500 Da) (PEG-DBCO).

A skilled artisan will appreciate that the invention is not limited to a 4-arm versions of PEG. Both linear and branched (3, 4, and 8 arm) PEG-amines of varying molecular weight are commercially available and are used as starting materials with the same procedure to attach the DBCO.

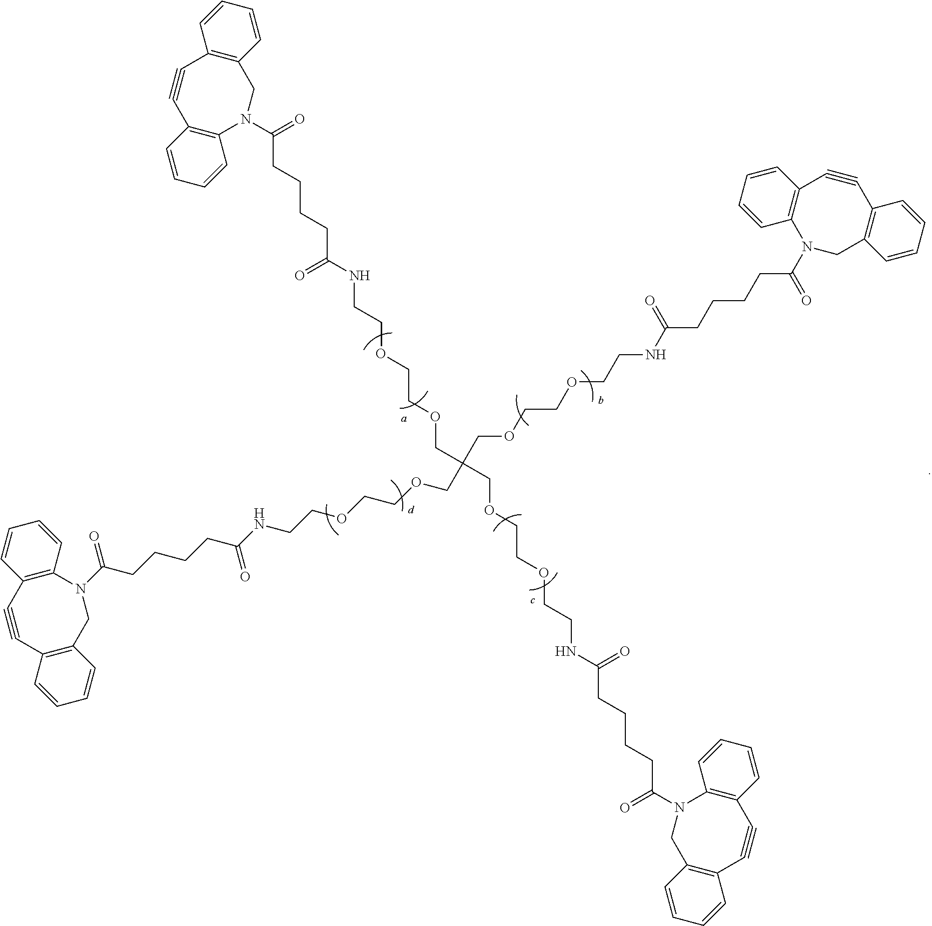

The structure above is a 4-arm poly(ethylene glycol) (PEG) tetradibenzocyclooctyne (4-arm-PEG-DBCO).

### Example 4. Alkaline Phosphatase Stabilization

In certain instances, the photodegradable composition in Structure V-A is the Azide-RGGRK(PLazide)-NH₂ and the other component of the hydrogel is the four-arm PEG-DBCO. The two components are reacted in a 2:1 azide-containing peptide:DBCO-containing PEG molar ratio to form a hydrogel within minutes at room temperature. In one example, a biologic, such as the enzyme alkaline phosphatase (ALP), is present in the reaction mixture. The hydrogel encapsulates the biologic, and upon vacuum drying at 4° C. most of the water will evaporate, leaving only water of hydration, the polymer and the biologic in solid form. The hydrogel, with the protected biologic, can now be stored at elevated temperature. After storage, the hydrogel is exposed to water and irradiated with 365 nm light (6 mW/cm²) for 10 min to degrade the hydrogel. The protected biologic is now released back in solution and we can verify temperature protection by activity assays. ALP has shown 60-80% retention of enzymatic activity after storage at 60° C. for four weeks. The protection of enzymatic activity depends on weight loading of biologic, specific formulation of invention and stabilization additives used.

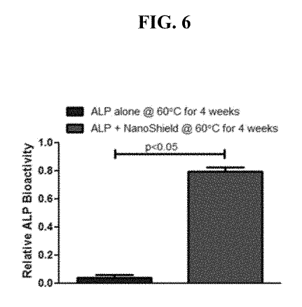

FIG. 6. shows the results of alkaline phosphatase (ALP) that was stored at 60° C. for four weeks, with and without the protection of the invention. Compared to a control sample stored at −70° C. retention of enzymatic activity was ˜5% for the sample stored without the invention and ˜80% after photorelease for the sample stored with the protection of the invention.

### Example 5. T4 DNA Ligase Stabilization

A biologic, such as the enzyme T4 DNA ligase (T4 or ligase), can be encapsulated, stored and stabilized by a photodegradable composition of the invention at different temperatures. For example, the photodegradable composition of V-A and the four-arm PEG-DBCO are reacted in a 2:1 azide-containing peptide:DBCO-containing-PEG molar ratio as described above to form a hydrogel, referred to as NanoShield. In general, the ligase encapsulated with NanoShield (i.e., NanoShield encapsulated samples) and the ligase not encapsulated with NanoShield (i.e., control samples) were stored at the indicated temperatures. Additionally, a NanoShield encapsulated ligase sample was stored at −20° C., which is the manufacturer's recommended storage temperature. The manufacturer of the T4 DNA ligase used herein is New England BioLabs (NEB).

All samples were prepared in clear thin-walled PCR strip tubes. After the indicated length of storage the samples were released into 50 μL of nuclease free water by exposure to UV light (λ=365 nm, Iₒ=10 mW/cm²) for 20 minutes. The ligase activity was detected by performing a ligation reaction on a HindIII digest of λ DNA, using the manufacturer's protocol. The reaction products were separated on a 1% TAE/agarose gel, and visualized by ethidium bromide staining.

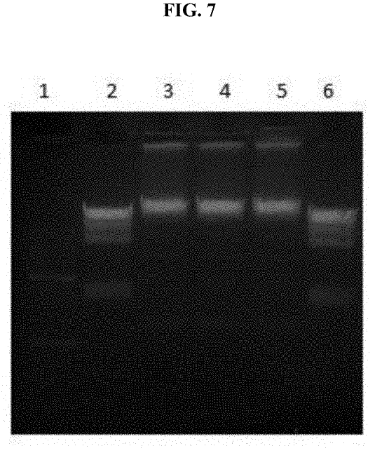

In one ligase stabilization experiment, ligase (NanoShield encapsulated samples and control samples) was stored for 24 hours at 40° C. in a thermocycler. This experiment was repeated a total of three times, with essentially identical results. Ligase is a highly thermally labile enzyme, and as such 40° C. was chosen for the initial studies. The results of the ligase stabilization study performed at 40° C. are shown in FIG. 7. In lane 2, which only contains DNA fragments, four bright bands are observed (FIG. 7). When active ligase is present, such as in lanes 3 to 5, the bands are reduced to one larger molecular weight band, which migrates through the gel more slowly. When ligase has been inactivated, as in lane 6 (control sample), the banding pattern is similar to no enzyme present. The gel shown in FIG. 7 indicates that NanoShield encapsulated ligase (lanes 4 and 5) remains completely active even after 24 hours at 40° C., while unencapsulated ligase (lane 6) is completely inactivated.

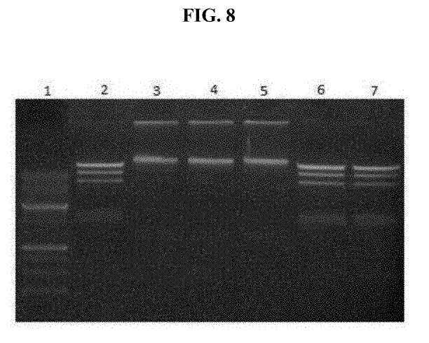

In another ligase stabilization experiment, NanoShield encapsulated ligase and control ligase samples were stored at 60° C. for 30 minutes in a thermocycler. This experiment was performed a total of three times. According to the manufacturer, T4 ligase is completely inactivated after 20 minutes at 60° C., which is confirmed by the results shown in FIG. 8 (lane 6). However, the enzymatic activity of the NanoShield encapsulated ligase is retained even after being stored at 60° C. for 30 minutes, as shown in lanes 4 and 5 of FIG. 8, which is longer than the length of time used for the manufacturer's recommended heat inactivation protocol.

### Example 6. Beta-Galactosidase Stabilization

The photodegradable composition of the invention is shown to also encapsulate and stabilize the beta-galactosidase (βGal) enzyme. The NanoShield hydrogel was prepared as described above and used to encapsulate βGal. Samples of βGal encapsulated with NanoShield (i.e., +NanoShield) and samples of βGal not encapsulated with NanoShield (i.e., −NanoShield) were stored at 60° C. for two weeks and for four weeks. After the indicated length of storage, the +NanoShield samples were exposed to UV light (λ=365 nm, Iₒ=10 mW/cm²) for 20 minutes, photoreleasing the βGal enzyme from encapsulation.

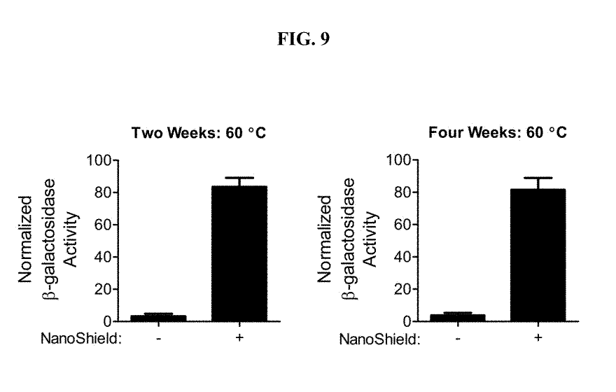

The βGal activity was determined by comparing performing a ligation reaction on a HindIII digest of λ DNA, using known protocols. The enzymatic activity of each βGal sample was measured by adding 50 μL of each βGal solution, +NanoShield (photoreleased) and −NanoShield, to a 100 μL solution of 16 mM ortho-nitrophenyl-β-galactoside (ONPG) in 100 mM phosphate buffer at pH 7.2. Using known protocols, the retention of βGal activity for each sample was determined by comparing the activity of a βGal control sample stored at 4° C. to the βGal samples stored at 60° C. for two weeks and four weeks. As shown in FIG. 9, only about 3% of the enzymatic activity was retained for the unprotected βGal samples, which were stored for two weeks at 60° C. and stored for four weeks at 60° C. (−NanoShield). Conversely, >80% of the enzymatic activity was retained for the protected βGal samples stored at 60° C., regardless of whether the samples were stored for two weeks or four weeks (+NanoShield).

### Example 7. Anti-CD3 Stabilization

The following example demonstrates the use of a photodegradable composition of the invention to encapsulate and stabilize the anti-CD3 antibody (UCHT1 LEAF, BioLegend, San Diego, Calif.) under various storage conditions. Samples of 1 mg/mL anti-CD3 antibodies encapsulated within NanoShield were prepared and subsequently dried as described above in Example 4. Un-encapsulated 1 mg/mL anti-CD3 antibody samples were suspended in Gibco PBS. Samples of dried NanoShield-encapsulated anti-CD3 and samples of non-dried anti-CD3 in PBS were stored at 65° C. for 1 hour and for 72 hours. After the indicated length of storage, the NanoShield samples were exposed to long-wave LED light (λ=365 nm, Iₒ=10 mW/cm²) for 20 minutes, photoreleasing the anti-CD3 antibody from the dried hydrogel. Temperature protection of anti-CD3 antibodies was verified by activity assays. The maximum response of the assays was determined by fresh anti-CD3 antibody at an equal concentration in the assay.

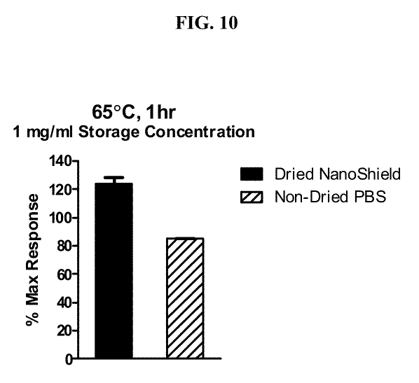

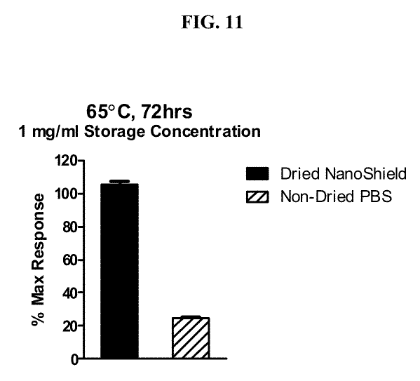

As shown in FIG. 10, when stored at 65° C. for 1 hour, about 80% of the anti-CD3 antibody maximum response was retained for the unprotected non-dried PBS anti-CD3 sample and 100% of the anti-CD3 max response was retained for the protected dried NanoShield anti-CD3 sample. FIG. 11 shows the storage stability results of the unprotected non-dried PBS anti-CD3 and the protected dried NanoShield anti-CD3 samples stored at 65° C. for 72 hours. Only about 25% of the anti-CD3 antibody maximum response was retained for the unprotected non-dried PBS anti-CD3 sample while 100% of the anti-CD3 max response was retained for the protected dried NanoShield anti-CD3 sample.

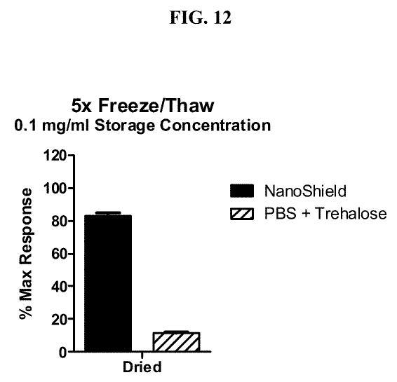

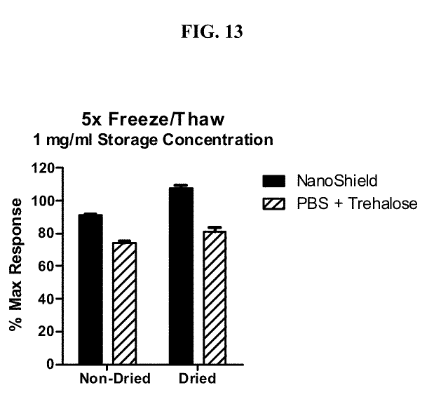

In another experiment, samples of encapsulated anti-CD3 (0.1 mg/mL) in dried NanoShield and un-encapsulated anti-CD3 (0.1 mg/mL) in dried PBS+5% Trehalose (industry standard excipients) were subjected to five freeze/thaw cycles (−20° C./25° C.). Then, the NanoShield samples were photoreleased from encapsulation and temperature protection of anti-CD3 antibodies was verified by activity assays as described previously. FIG. 12 shows the freeze/thaw stability results of the dried samples. Only about 15% of the anti-CD3 antibody maximum response was retained for the unprotected dried PBS+Trehalose anti-CD3 sample while >80% of the anti-CD3 max response was retained for the protected dried NanoShield anti-CD3 sample. In a similar experiment, samples of encapsulated anti-CD3 (1 mg/mL) in dried and non-dried NanoShield and un-encapsulated anti-CD3 (1 mg/mL) in dried and non-dried PBS+Trehalose were subjected to five freeze/thaw cycles (−20° C./25° C.). The protected samples were photoreleased from NanoShield, and the protected and un-protected anti-CD3 antibodies were samples stored as dried samples and non-dried samplers were assayed for activity. FIG. 13 shows the freeze/thaw stability results of the non-dried and dried samples. About 90% of the anti-CD3 max response was retained for the protected non-dried NanoShield anti-CD3 sample and about 75% of the anti-CD3 antibody maximum response was retained for the unprotected non-dried PBS+Trehalose anti-CD3 sample (FIG. 13). As shown in FIG. 13, 100% of the anti-CD3 max response was retained for the protected dried NanoShield anti-CD3 sample and about 80% of the anti-CD3 antibody maximum response was retained for the unprotected dried PBS+Trehalose anti-CD3 sample.

### Example 8. Ovalbumin Stabilization and In Vivo Immunization

The following example demonstrates the use of a photodegradable composition of the invention to encapsulate and stabilize ovalbumin (Sigma-Aldrich Corp., St. Louis, Mo.) at elevated temperatures for in vivo testing. Four different ovalbumin (OVA) samples were prepared and stored at elevated temperatures, and injected into Balb/C mice as immunizations to determine whether NanoShield could protect the immunogenicity of antigens that had been exposed to elevated temperatures. NanoShield encapsulated OVA samples were prepared as described above; one was stored at 60° C. for 7 days and the second NanoShield encapsulated OVA sample was stored for 14 days at 60° C. Un-encapsulated OVA samples were also prepared and stored at 60° C. for 7 days and for 14 days. After storage, protected OVA samples were released from NanoShield encapsulation into sterile PBS using the same light conditions as described in Example 7. Five immunizations were prepared: four immunizations using the stressed OVA samples (60° C. for 7 days with or without NanoShield and 60° C. for 14 days with or without NanoShield), and one freshly prepared OVA immunization with alum (40 μg/injection) at a final volume of 200 μL in sterile PBS. Groups of 5 mice were injected i.p. with the ova immunizations. A group of untreated mice was used as the control group. Serum was collected weekly for 6 weeks via r.o. bleeds for each mouse group. IgG1 titers were determined by ELISA (minimum dilution 1:400).

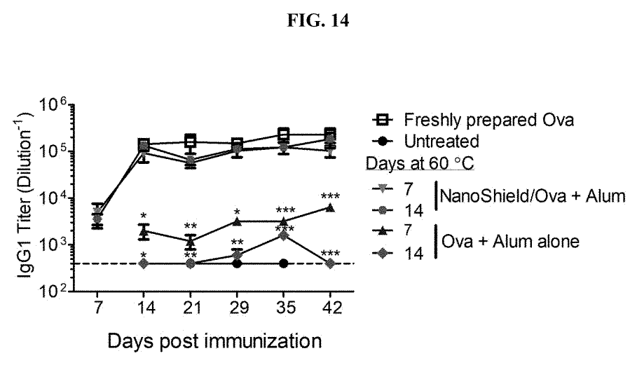

As shown in FIG. 14, the mice that received OVA immunizations prepared from the NanoShield protected samples had the highest IgG1 titers of about 10⁵ even 42 days post immunization, regardless of whether the samples were stored for 7 days or 14 days. Conversely, the mice that received OVA immunizations prepared from the un-protected samples had >10³ IgG1 serum levels (un-protected OVA, stored for 7 days) and >10² IgG1 serum levels (un-protected OVA, stored for 14 days), the latter of which more or less matches the IgG1 levels for the untreated mice (control group).

### Example 9. Encapsulation and Stabilization of Influenza Vaccines

The following example prophetically demonstrates the use of a photodegradable composition of the invention to encapsulate and stabilize the influenza vaccine. The NanoShield hydrogel was prepared as described above in the presence of the influenza vaccine, Agrippal, encapsulating the biologic. After encapsulation, samples of the vaccine-encapsulated-hydrogel are vacuum dried as described above leaving only waters of hydration, the polymer and the vaccine in solid form. Samples of the hydrogel, with the protected influenza vaccine, are stored at an elevated temperature of 60° C. for four weeks. Samples of the unprotected influenza vaccine are also stored at 60° C. for four weeks. As a control, one sample of the protected influenza vaccine and one sample of the unprotected vaccine are both stored at 5° C., the recommended storage temperature, for four weeks.

After storage, the hydrogel is exposed to a vaccine appropriate diluent and irradiated with 365 nm light (6 mW/cm²) for 10 min to degrade the hydrogel and release the protected influenza vaccine in solution. The efficacy of stabilization provided to the encapsulated influenza vaccine by the photodegradable composition in vivo will be determined by the hemagglutination inhibition (HAI) assay performed on samples collected from immunized mice or ferrets, the methods of which are well known to those skilled in the art. See, e.g., Noah, D. L., et al. “Qualification of the Hemagglutination Inhibition Assay in Support of Pandemic Influenza Vaccine Licensure.” Clinical and Vaccine Immunology. 16:4 (2009) 558-66. Using known protocols, the retention of the vaccine's efficacy for each sample is measured by comparing the activity of the vaccine control samples stored at 5° C. to the activity of the protected and unprotected vaccine samples stored at 60° C. for four weeks. The activity of the unprotected vaccine stored for four weeks at 60° C. will be about from 0-15%, while the activity of the protected vaccine stored for four weeks at 60° C. will be about from 60-90%.

Although the description herein contains many specificities, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference herein to provide details concerning additional starting materials, additional methods of synthesis, additional methods of analysis and additional uses of the invention.

What is claimed is:

1. A photodegradable composition, the photodegradable composition comprising: one or more photolabile nitrobenzo groups having the formula: 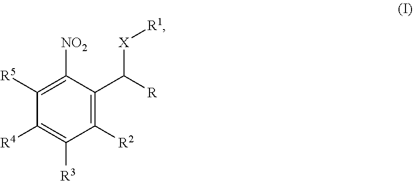
wherein the one or more photolabile nitrobenzo groups comprises a polymeric backbone structure and a linker through which the backbone structure is attached to the photolabile nitrobenzo group;
X is a member selected from the group consisting of O, NH and S;
R is a member selected from the group consisting of hydrogen, a straight-chain or branched C₁-C₁₀ alkyl, aryl, alkoxy, aryloxy, or a carboxy group in which one or more carbon atoms can be independently optionally substituted with one or more heteroatoms, and one or more hydrogen atoms can be independently optionally substituted with hydroxyl, halogen, an oxygen atom, or backbone structure having at least one reactive end group, where the backbone structure can be appended through a peptide linker or a PEG linker, where the linker can have at least one reactive end group;
at least one of R¹, R², R³, R⁴, or R⁵, is a backbone structure comprising at least one reactive end group, wherein the backbone structure is appended through a peptide linker or a PEG linker, where the linker comprises at least one reactive end group;
R¹, R², R³, R⁴, or R⁵ are each independently selected from the group consisting of hydrogen; straight chain, branched or cyclic C₁-C₂₀ alkyl, alkenyl, alkynyl groups in which one or more of the carbon atoms are optionally substituted with non-hydrogen substituents and wherein one or more C, CH or CH₂ moiety can be replaced with an oxygen atom, a nitrogen atom, an —NR′ group, a —CO—R′ group, a S atom, or a reactive end group;
one or more R¹, R², R³ R⁴, or R⁵ can be optionally substituted with one or more substituents selected from halogen; nitro; cyano; isocyano; thiocyano; isothiocyano; azide; —SO₂; —OSO₃H; one or more optionally substituted straight-chain, branched or cyclic alkyl, alkenyl or alkynyl groups; OR′; —CO—OR′; —O—CO—R′; —N(R′)₂; —CO—N(R′)₂; —NR′—CO—OR′; —SR′; —SOR′; —SO₂—R′; —SO₃R′; —SO₂N(R′)₂; —P(R′)₂; —OPO₃(R′)₂; and —Si(R′)₃, wherein each R′, independent of other R′ in the substituent group can be a hydrogen, an optionally substituted straight-chain, branched or cyclic alkyl, alkenyl or alkynyl group wherein one or more C, CH or CH₂ groups therein can be replaced with an O atom, N atom, S atom or —NH group; an optionally substituted aromatic group, two or more R′ groups can be linked together to form a ring which may contain one or more of the same or different heteroatoms; and
R′ can in turn be optionally substituted with one or more groups selected from the group consisting of halogens, reactive end groups, nitro groups; cyano groups; isocyano groups; thiocyano groups; isothiocyano groups; azide groups; —SO₂ groups; —OSO₃H groups; straight-chain, branched or cyclic alkyl, alkenyl or alkynyl groups; halogenated alkyl groups; hydroxyl groups; alkoxy groups; carboxylic acid and carboxylic ester groups; amine groups; carbamate groups, thiol groups, thioether and thioester groups; sulfoxide groups, sulfone groups; sulfide groups; sulfate and sulfate ester groups; sulfonate and sulfonate ester groups; sulfonamide groups, sulfonate ester groups; phosphine groups; phosphate and phosphate ester groups; phosphonate and phosphonate ester groups; and alkyl-substituted silyl groups.

2. The photodegradable composition of claim 1, wherein R¹ is a —CO—R′ group and R′ is a substituted alkyl having a reactive end group, wherein the reactive end group is an azide group or an alkynyl group.

3. The photodegradable composition of claim 1, wherein R² and R⁵ are each hydrogen.

4. The photodegradable composition of claim 1, wherein R³ is a member selected from the group consisting of an alkoxy and substituted alkoxy, wherein the substituent is a carboxylic acid.

5. The photodegradable composition of claim 1, wherein R⁴ is a member selected from the group consisting of an alkoxy and substituted alkoxy, wherein the substituent is a carboxylic acid.

6. The photodegradable composition of claim 1, wherein the one or more photolabile nitrobenzo groups is a photolabile nitrobenzo group having a formula selected from the group consisting of:

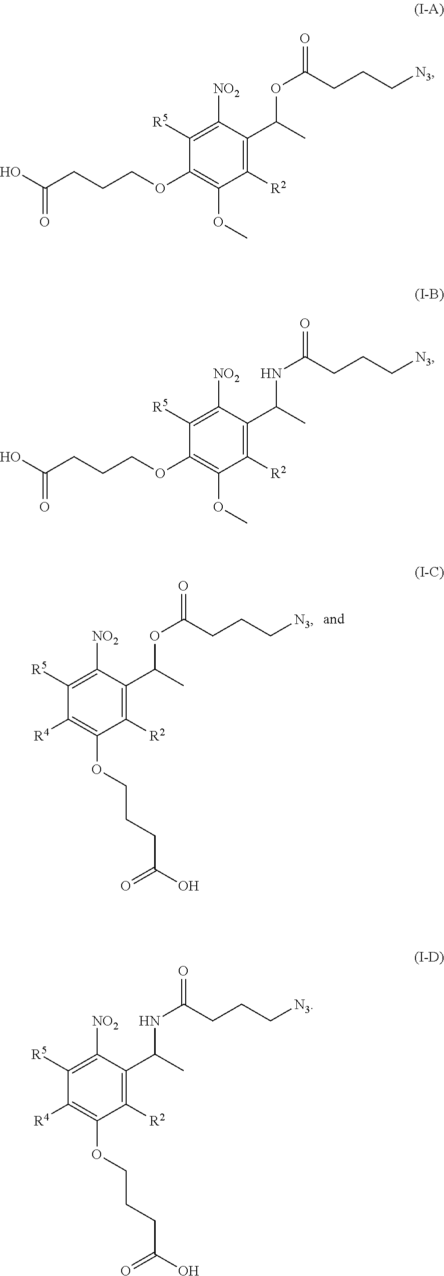

7. The photodegradable composition of claim 1, wherein the linker is a peptide linker comprising amino acid monomers, wherein the amino acid monomers are members selected from the group consisting of A, C, D, E, F, G, H, I, K, L, M, N, P, Q, R, S, T, V, W, and combinations thereof.

8. The photodegradable composition of claim 7, wherein peptide linker is:
-RGGRK-,
wherein the N terminus comprises a reactive group and the C terminus comprises an amine.

9. The photodegradable composition of claim 8, wherein the photodegradable composition comprises the following peptide linker and the following photolabile nitrobenzo group, having the formula:

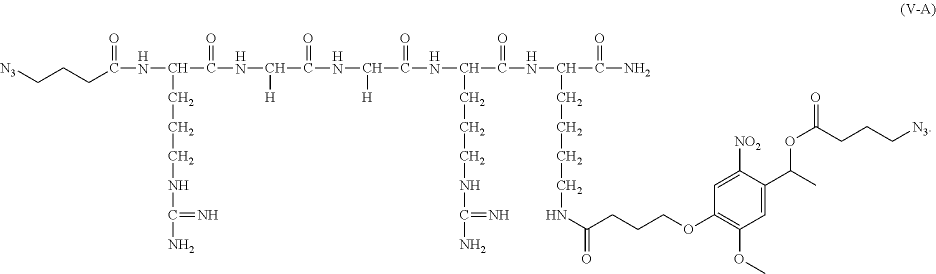 [SMILES: COc1cc(C(C)OC(=O)CCCN=[N+]=[N-])c([N+](=O)[O-])cc1OCCCC(=O)NCCCCC(NC(=O)C(CCCNC(=N)N)NC(=O)CNC(=O)CNC(=O)C(CCCNC(=N)N)NC(=O)CCCN=[N+]=[N-])C(N)=O]

10. The photodegradable composition of claim 1, wherein the polymeric backbone structure is a multi-arm polyethylene glycol PEG polymer functionalized with dibenzocyclooctyne (DBCO) reactive groups.

11. The photodegradable composition of claim 10, wherein the multi-arm polyethylene glycol PEG polymer has the following formula: 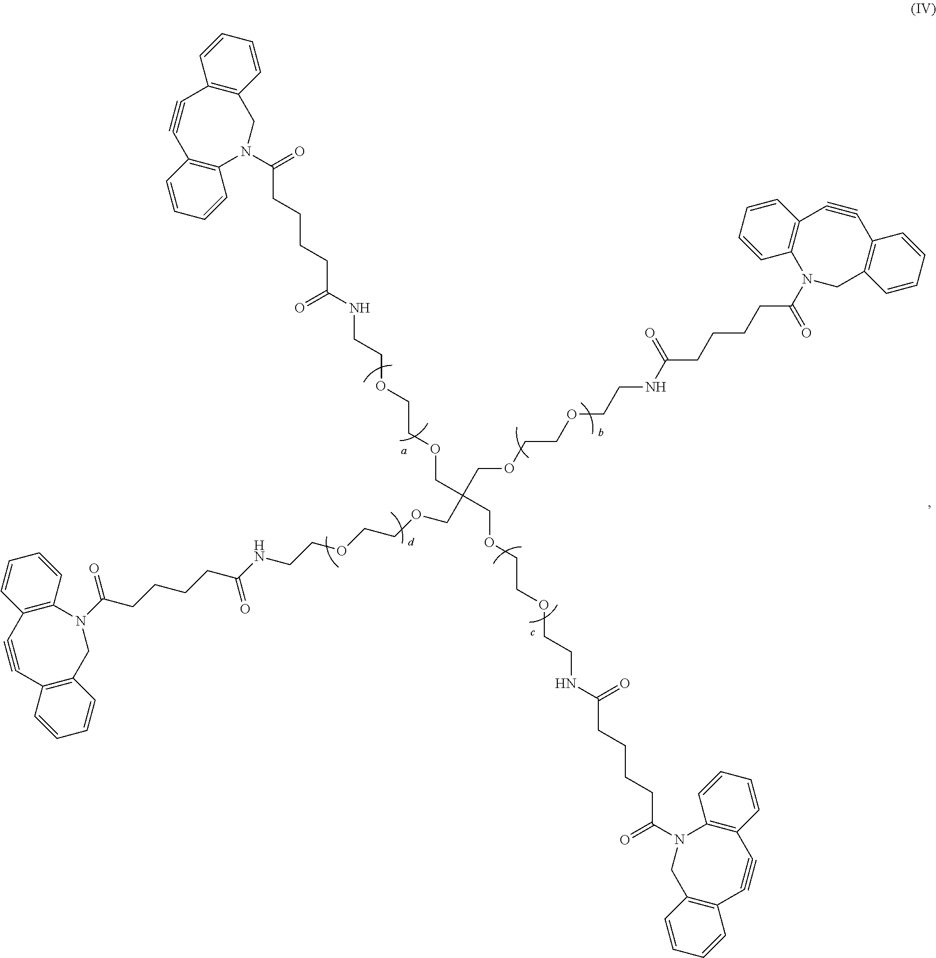
wherein a, b, c, and d are each independently selected from 1 to 200.

12. The photodegradable composition of claim 9, wherein the photodegradable composition comprises reacting the compound of formula (V-A) with a multi-arm polyethylene glycol PEG polymer of formula (IV).

13. The photodegradable composition of claim 6, wherein the composition has an entrapped biomolecule, which is releasable upon photodegradation of the composition.

14. The photodegradable composition of claim 13, wherein the composition is photodegraded with light irradiation at a wavelength of from about 200 nm to about 500 nm, thereby releasing the entrapped biomolecule.

15. The photodegradable composition of claim 13, wherein the composition is photodegraded with light irradiation at a wavelength of from about 390 nm to about 850 nm, thereby releasing the entrapped biomolecule.

16. The photodegradable composition of claim 13, wherein the entrapped biomolecule is a member selected from the group consisting of a protein, a peptide, an enzyme, an enzyme substrate, a vaccine, a hormone, an antibody, an antibody fragment, an antigen, a hapten, an avidin, a streptavidin, a carbohydrate, an oligosaccharide, a polysaccharide, a nucleic acid, a fragment of DNA, a fragment of RNA and a biological therapeutic.

17. A photodegradable composition, the photodegradable composition comprising: one or more coumarin-azide cinnamic photorelease groups having the formula: 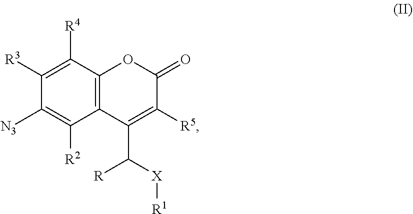
wherein the one or more coumarin-azide cinnamic photorelease groups comprises a polymeric backbone structure and a linker through which the backbone structure is attached to the coumarin-azide cinnamic photorelease group;
X is a member selected from the group consisting of O, NH and S;
R is a member selected from the group consisting of hydrogen, a straight-chain or branched C₁-C₁₀ alkyl, aryl, alkoxy, aryloxy, or a carboxy group in which one or more carbon atoms can be independently optionally substituted with one or more heteroatoms, and one or more hydrogen atoms can be independently optionally substituted with hydroxyl, halogen, an oxygen atom, or backbone structure having at least one reactive end group, where the backbone structure can be appended through a peptide linker or a PEG linker, where the linker can have at least one reactive end group;
at least one of R¹, R², R³, R⁴, or R⁵, is a backbone structure comprising at least one reactive end group, wherein the backbone structure is appended through a peptide linker or a PEG linker, where the linker comprises at least one reactive end group;
R¹, R², R³, R⁴, or R⁵ are each independently selected from the group consisting of hydrogen; straight chain, branched or cyclic C₁-C₂₀ alkyl, alkenyl, alkynyl groups in which one or more of the carbon atoms are optionally substituted with non-hydrogen substituents and wherein one or more C, CH or CH₂ moiety can be replaced with an oxygen atom, a nitrogen atom, an —NR′ group, a —CO—R′ group, a S atom, or a reactive end group;
one or more R¹, R², R³ R⁴, or R⁵ can be optionally substituted with one or more substituents selected from halogen; nitro; cyano; isocyano; thiocyano; isothiocyano; azide; —SO₂; —OSO₃H; one or more optionally substituted straight-chain, branched or cyclic alkyl, alkenyl or alkynyl groups; OR′; —CO—OR′; —O—CO—R′; —N(R′)₂; —CO—N(R′)₂; —NR′—CO—OR′; —SR′; —SOR′; —SO₂—R′; —SO₃R′; —SO₂N(R′)₂; —P(R′)₂; —OPO₃(R′)₂; and —Si(R′)₃, wherein each R′, independent of other R′ in the substituent group can be a hydrogen, an optionally substituted straight-chain, branched or cyclic alkyl, alkenyl or alkynyl group wherein one or more C, CH or CH₂ groups therein can be replaced with an O atom, N atom, S atom or —NH group; an optionally substituted aromatic group, two or more R′ groups can be linked together to form a ring which may contain one or more of the same or different heteroatoms; and
R′ can in turn be optionally substituted with one or more groups selected from the group consisting of halogens, reactive end groups, nitro groups; cyano groups; isocyano groups; thiocyano groups; isothiocyano groups; azide groups; —SO₂ groups; —OSO₃H groups; straight-chain, branched or cyclic alkyl, alkenyl or alkynyl groups; halogenated alkyl groups; hydroxyl groups; alkoxy groups; carboxylic acid and carboxylic ester groups; amine groups; carbamate groups, thiol groups, thioether and thioester groups; sulfoxide groups, sulfone groups; sulfide groups; sulfate and sulfate ester groups; sulfonate and sulfonate ester groups; sulfonamide groups, sulfonate ester groups; phosphine groups; phosphate and phosphate ester groups; phosphonate and phosphonate ester groups; and alkyl-substituted silyl groups.

18. The photodegradable composition of claim 17, wherein the linker is a PEG linker having a molecular weight of about 1 kDa to about 40 kDa.

19. The photodegradable composition of claim 17, wherein the linker is a peptide linker comprising amino acid monomers, wherein the amino acid monomers are members selected from the group consisting of A, C, D, E, F, G, H, I, K, L, M, N, P, Q, R, S, T, V, W, and combinations thereof.

20. The photodegradable composition of claim 19, wherein peptide linker is:
-RGGRK-,
wherein the N terminus comprises a reactive group and the C terminus comprises an amine.
